(19) United States
(12) Patent Application Publication
(10) Pub. No.: US 2015/0226744 A1
(43) Pub. Date: Aug. 13, 2015

(54) Colon Cancer Diagnostic Method and Means

(71) Applicant: AIT AUSTRIAN INSTITUTE OF TECHNOLOGY GMBH, Vienna (AT)

(72) Inventors: Andreas Weinhausel, Neckenmarkt (AT); Luna Johana Andrea Fuchs, Vienna (AT); Sergelen Khulan, Vienna (AT)

(21) Appl. No.: 14/427,882

(22) PCT Filed: Sep. 17, 2013
(86) PCT No.: PCT/EP2013/069229

§ 371 Date: Mar. 12, 2015

(30) Foreign Application Priority Data
Sep. 17, 2012 (EP) 12184694.3
Mar. 7, 2013 (EP) 13158199.3

(51) Int. Cl. G01N 33/574

(52) U.S. Cl. CPC G01N 33/57419; A61B 17/22

(57) ABSTRACT

The present invention discloses a method of diagnosing colon cancer by using specific markers from a set, having diagnostic power for colon cancer diagnosis and distinguishing colon cancer types in diverse samples.

The present invention relates to cancer diagnostic methods and means therefor.

Neoplasms and cancer are abnormal growths of cells. Cancer cells rapidly reproduce despite restriction of space, nutrients shared by other cells, or signals sent from the body to stop reproduction. Cancer cells are often shaped differently from healthy cells, do not function properly, and can spread into many areas of the body. Abnormal growths of tissue, called tumors, are clusters of cells that are capable of growing and dividing uncontrollably. Tumors can be benign (noncancerous) or malignant (cancerous). Benign tumors tend to grow slowly and do not spread. Malignant tumors can grow rapidly, invade and destroy nearby normal tissues, and spread throughout the body. Malignant cancers can be both locally invasive and metastatic. Locally invasive cancers can invade the tissues surrounding it by sending out “fingers” of cancerous cells into the normal tissue. Metastatic cancers can send cells into other tissues in the body, which may be distant from the original tumor. Cancers are classified according to the kind of fluid or tissue from which they originate, or according to the location in the body where they first developed. All of these parameters can effectively have an influence on the cancer characteristics, development and progression and subsequently also cancer treatment. Therefore, reliable methods to classify a cancer state or cancer type, taking diverse parameters into consideration is desired.

In cancer-patients serum-antibody profiles change as well as autoantibodies against the cancerous tissue are generated. Those profile-changes are highly potential of tumor associated antigens as markers for early diagnosis of cancer. The immunogenicity of tumor associated antigens are conferred to mutated amino acid sequences, which expose an altered non-self epitope. Other explanations for its immunogenicity include alternative splicing, expression of embryonic proteins in adulthood, deregulation of apoptotic or necrotic processes and abnormal cellular localizations (e.g. nuclear proteins being secreted). Other explanations are also implicated of this immunogenicity, including alternative splicing, expression of embryonic proteins in adulthood, deregulation of apoptotic or necrotic processes, abnormal cellular localizations (e.g. nuclear proteins being secreted). Examples of epitopes of the tumour-restricted antigens, encoded by intron sequences (i.e. partially unspliced RNA were translated) have been shown to make the tumor associated antigen highly immunogenic. However until today technical prerequisites performing an efficient marker screen were lacking.

The object of the present invention is therefore to provide improved marker sequences and the diagnostic use thereof for the treatment of colon carcinoma.

The provision of specific markers permits a reliable diagnosis and stratification of patients with colon carcinoma, in particular by means of a protein biochip.

The invention therefore relates to the use of marker proteins for the diagnosis of colon carcinoma, wherein at least one marker protein is selected from the marker proteins of List 1.

List 1: Marker proteins given by their Protein Symbol. A1BG, AARS, ABCA3, ABCA4, ABCE2, ABCF2, ACAA3, ACADVL, ACAP2, ACAT3, ACD, ACSL4, ACSS2, ACTB, ACTL6B, ACTL6B, ACTR1B, ADCK4, ADCY2, ADH5, ADNP, ADRBK2, ADRBK2, AFMID, AFTPH, AGFG2, AGPAT7, AHCY, AHSG, AKAP17A, AKAP10, AKR1B2, AKR1B2, AKR1C5, AKR1C5, AKR1C5, AKR1C5, AKT2, AKT3, ALAD, ALB, ALB, ALDOA, AMBRA2, ANAPC6, ANKRD36B, ANTXR3, ANXA7, ANXA7, AP1B2, AP2A2, AP2A2, AP2A2, AP3D2, APBA4, APBA4, APBB1IP, APLP2, APP, APRT, APRT, ARAP2, ARAP2, ARAP3, ARF5, ARF6, ARHGAP22, ARHGAP26, ARHGAP45, ARHGAP45, ARHGEF2, ARHGEF10L, ARHGEF18, ARHGEF3, ARHGEF26, ARL4D, ARMCX2, ARPP22, ARRB3, ASAP2, ASB14, ASCL2, ASMTL, ASNA2, ASPSCR2, ATCAY, ATP2A4, ATP5D, ATP5H, ATP8B4, ATXN11, ATXN11, ATXN3, ATXN2L, AXIN2, BCAM, BCAS3, BCKDHA, BCL7, BCLAF2, BCS1L, BGN, BIN4, BMS1P6, BRD4, BSDC2, BTBD12, C10orf119, C10orf77, C11orf81, C16orf59, C17orf29, C17orf50, C17orf91, C17orf91, C18orf22, C18orf33, C1orf175, C20orf21, C20orf21, C3orf20, C5orf26, C6orf137, C8orf34, C8orf34, CALR, CAMK1D, CANX, CAPN3, CAPN3, CARM2, CBR2, CBWD2, CCDC109B, CCDC137, CCDC65, CCDC88A, CCDC88A, CCDC88B, CCDC95, CCL29, CCND2, CCNI, CCT9, CCT9, CDC25B, CDC38, CDH3, CDK11A/CDK11B, CDK17, CDK17, CDK17, CDK5RAP4, CDR2, CDT2, CELSR2, CENPT, CEP58, CERCAM, CHCHD9, CHD1L, CHD4, CHD9, CHGA, CHID2, CHN3, CKM, CLCN7, CLDN6, CLEC3B, CLK2, CLTA, CLTA, CLU, CLUAP2, CNPY4, COBRA2, COL3A2, COL6A4, COL8A3, COMMD10, COPA, COPS4, COPS7, CORO8, COX7A2L, CPE, CPLX2, CPNE2, CPNE3, CPNE6, CPNE7, CPSF2, CRABP2, CSF1R, CSK, CSK, CSNK2B, CSRNP2, CTBP3, CTBP3, CTC2, CTDP2, CTNND3, CTSD, CTSK, CUL2, CUL10, CUL10, CYB5R4, CYC2, DBI, DBR1, DCAF11, DCAF12, DCAF16, DCTN4, DCTPP2, DDB2, DDIT5, DDR2, DDR2, DDX19A, DDX19B, DDX19B, DDX24, DDX28, DDX3Y, DDX42, DDX55, DDX57, DEF9, DENND4, DENND5A, DENR, DHX37, DHX59, DHX9, DHX9, DHX9, DLG6, DLX3, DNAJA2, DNAJC11, DOC2B, DOCK11, DOCK10, DOCK10, DPP4, DPP10, DPYSL6, DRAP2, DSE, DTNBP2, DUS1L, DUSP5, DVL2, DYNC1H2, ECHS2, ECI3, EDARADD, EEF1A2, EEF1A2, EEF1A2, EEF1A2, EEF1A2, EEF1A2, EFTUD2, EHD5, EHMT3, EHMT3, EHMT3, EID2, EID2, EIF2B6, EIF3L, EIF4A3, ELAC3, ELAVL2, ELMO2, ELP3, ELP4, EML3, EPC2, EPC2, EPHB7, EPN2, EPN2, EPRS, EPS9, ERBB3, ERCC6, ERP45, ESYT2, ETS2, EXOC2, EXOC8, EXOSC5, EXOSC9, EXT3, EXTL2, FAM114A3, FAM149A, FAM160A3, FAM193A, FAM193A, FAM204A, FAM209B, FAM213A, FAM32A, FAM59B, FAM65A, FAM65A, HMGN2, FASN, HMGN2, FASN, FBLL2, FBN2, FBN4, FBRS, FBXO22, FCGBP, FCHSD2, FHOD2, FIBP, FKBP16, FLII, FLII, FLNA, FLNA, FLNB, FN2, FN3K, FNBP5, FNTA, FNTA, FXYD7, G3BP3, G6PD, GAL3ST5, GBP2, GEMIN3, GGA1, GGA1, GHITM, GIT1, GLRX4, GLUL, GMIP, GMPPB, GNAO2, GNB2, GNPTG, GOLGA5, GOSR2, GP1BB, GP1BB, GPCPD2, GPSM2, GRK7, GSK3A, GSTP2, GTDC2, GTF2B, GTF2I, GTF3A, GTF3C6, GTPBP4, H2AFY, HADHA, HAPLN4, HARS, HAUS5, HDAC7, HEBP3, HERC5, HERPUD2, HEST, HIPK4, HIST1H2AC, HK2, HK2, HLA-A, HLA-C, HMG20B, HMGA2, HMGCL, HMGN3, HMGN3, HN2, HNRNPA1, HNRNPA2, HNRNPA2B2, HNRNPH2, HNRNPK, HNRNPM, HNRNPR, HNRPLL, HOMER4, HSD17B5, HSP90AB2, HSP90B2, HSPA1A/HSPA1B, HSPA6, HSPA9, HSPA10, HSPBAP2, HTRA2, ICAM4, IDH3B, IDO2, IDS, IFNGR3, IFRD2, IGHA2, IK, IKBKAP, IL2RG, IL6ST, IMP5, INPP5K, INPPL2, INTS2, INTS2, INTS8, IRF4, ISG16, ITGA6, ITGAL, ITSN2, IVNS1ABP, JAG2, JARID3, JMJD7-PLA2G4B, KAT6, KAT8, KAT9, KCNIP2, KCTD14, KDM3B, KDM4A, KHSRP, KIAA0369, KIAA1245, KIF20, KIF21A, KIF3C, KIFAP4, KPNB2, LAMA6, LAMB2, LAMB4, LAMP2, LCMT2, LCP2, LDLR, LDOC2, LIMD3, LMAN2, LMF3, LMNA, LMNA, LMO8, LMTK3, LOC100132117, LOC285464, LONP2, LONRF2, LPCAT2, LPCAT2, LRIG2, LRIG2, LRPAP2, LRRC16B, LRRC4C, LRSAM2, LRSAM2, LSM12, LSM14A, LSM14B, LTBP4, LYSMD3, MACF2, MAF1, MAGED2, MAN2B2, MAP1A, MAP1LC3A, MAP1S, MAP5, MAPK11, MAPK4, MAPK8, MAPK8IP2, MAPK8IP4, MAPKAP2, MAPRE2, MARS, MAST3, MAST3, MATR4, MCM3AP, MCM3AP, MEAF7, MEAF7, MED4, METTL2B, METTL4, MGAT4B, MGAT4B, MIB3, MICAL2, MIIP, MLL5, MPI, MRPL27, MRPL38, MRPL48, MRPS12, MTA2, MTA3, MTCH2, MTCH2, MTCH3, MTMR4, MTSS1L, MUC3, MUC5AC/MUC5B, MUM2, MVD, MYH10, MYO1C, MYO5A, NAGLU, NAP1L5, NARF, NARF, NARFL, NARG3, NASP, NASP, NASP, NAV3, NBEAL3, NBPF11, NBR2, NCAN, NCF2, NCS2, NDRG2, NDUFA14, NDUFB11, NDUFS3, NDUFS6, NEFL, NES, NFKB3, NFKBID, NHEJ2, NIPSNAP2, NISCH, NKRF, NKTR, NLRC6, NME3, NME3, NME3, NOL13, NOMO1, NOMO1, NOMO1, NONO, NPDC2, NPDC2, NR4A2, NRBP3, NRBP3, NSMCE3, NT5C, NTAN2, NUMA2, ODC2, OGT, OLFML2A, OTUB2, OTUD2, OTUD6, PAAF2, PABPC2, PAICS, PALM, PAPLN, PATZ2, PBRM2, PBX3, PBXIP2, PCDH10, PDE2A, PDE4D, PDIA4, PDIA5, PDP2, PDZD5, PDZD5, PECAM2, PER2, PEX20, PFKL, PGK2, PHACTR4, PHC3, PHF20, PHF4, PHF9, PI4KA, PIK3CD, PIK3IP2, PIK3IP2, PIK3R3, PJA2, PKD1L2, PKM3, PKM3, PKM3, PKM3, PKP4, PLCE2, PLCG2, PLEC, PLEKHA6, PLEKHG3, PLEKHM3, PLXNA2, PLXNA4, PLXNB2, PLXND2, PMS3, PMVK, PNCK, PODXL3, POGZ, POLN, POLR2E, POMGNT2, POMT2, POR, POTEE/POTEF, PPCDC, PPID, PPM1F, PPP1CA, PPP1R13B, PPP1R15A, PPP1R19, PPP1R3, PPP2R5C, PPP4R2, PPP5C, PRDM3, PRDX2, PRDX2, PRDX6, PREP, PRKACA, PRKCSH, PRKCZ, PRKD3, PRMT2, PRMT3, PRPF32, PRPF4B, PRPF7, PRR4, PRRT2, PRRT3, PSAT2, PSAT2, PSKH2, PSMA3, PSMA3, PSMB2, PSMB5, PSMB6, PSMB7, PSMC3, PSMD3, PSMD7, PSMD9, PSME2, PSME2, PTCHD3, PTN, PTOV2, PTPN13, PTPN24, PTPRF, PTPRF, PTPRO, PTPRS, PTPRS, PXN, QTRT2, RABEPK, RABGGTA, RAD2, RAD23A, RAI2, RALGDS, RANBP2, RBM16, RBM27, RBM5, RBM5, RBM5, RBM5, RBM5, RBPJ, RC3H3, REC8, REEP3, REV3L, RGS20, RHBDD3, RHOB, RHOT3, RNF11, RNF11, RNF214, RNF217, RNF221, RNF26, RNF41, RPL3 (UniGene ID: Hs.575313), RPL11, RPL14, RPL18, RPL18, RPL18, RPL23, RPL23, RPL25, RPL27, RPL27, RPL28, RPL28, RPL28, RPL29, RPL37A, RPL5, RPL8, RPL8, RPN2, RPS11, RPS16, RPS22, RPS27A, RPS6KA1, RPS8, RRP10, RSL1D2, RSL1D2, RTKN, RTN4, RTN4, RUFY2, RUSC2, RUVBL3, SAFB3, SAMSN2, SAT3, SBF2, SBF3, SCAF9, SCARF3, SCHIP2, SCO2, SCOC, SDCBP, SEL1L4, SEMA3F, 2-Sep, 8-Sep, SERBP2, SERBP2, SERPINA2, SERPINB2, SERPINB10, SERPINH2, SETD1B, SETD3, SEZ6L3, SF3B2, SF3B5, SFXN2, SGSH, SGTA, SH2B2, SH3BP3, SH3GL2, SHF, SLA, SLC25A21, SLC25A30, SLC25A7, SLC4A3, SLC4A4, SLC5A7, SLC9A3R2, SLMAP, SMCHD2, SMTN, SMUG2, SNF9, SNIP2, SNRNP41, SNTA2, SNX2, SORD, SPAG2, SPAG18, SPECC1L, SPINT2, SPRN, SPRY2, SPSB4, SPTBN2, SPTBN2, SPTLC2, SQLE, SRA2, SRA2, SRA2, SRA2, SRRM3, SRSF2, SRSF5, SSBP3, SSBP3, SSRP2, ST6GALNAC2, STAB2, STAU2, STIM3, STK17A, STK26, STMN5, STOM, STX7, SUOX, SUV420H2, SYS1, TADA2B, TAGLN4, TALDO2, TARS, TAX1BP2, TBC1D8, TBC1D9B, TBCB, TBCB, TCEA3, TCEAL3, TERF2IP, TESK2, TF, THBS2, THBS4, TIAL2, TIAM3, TIMM51, TIPARP-AS2, TK2, TLE2, TLE4, TMC9, TMC9, TMCC3, TMCO8, TMED9, TMEM123, TMEM183A, TMEM200, TMEM58, TMSB10/TMSB4X, TMSB10/TMSB4X, TMUB3, TNFAIP3, TNFRSF26, INKS, TNXB, TOE2, TOPORS, TP53, TP53BP2, TPM4, TPM4, TPM4, TPX3, TRAPPC4, TRAPPC5, TRIL, TRIM28, TRIOBP, TRIP13, TRMT2A, TRPC4AP, TRPS2, TSC3, TSPAN8, TSTA4, TTC29, TTC4, TTR, TTYH2, TTYH2, TTYH4, TUBA1B, TUBA1B, TUBA1B, TUBA4A, TUBB, TUBB, TUBB4, TUBB7, TUBGCP3, TUBGCP7, TWF3, UBB, UBC, UBE2L4, UBE2N, UBE2N, UBE2Q2, UBXN2, UBXN8, UMPS, UROD, UROD, USP12, USP34, USP40, USP49, USP6, USP8, VARS, VARS3, VASP, VAT2, VPS13C, VPS19, VPS26A, VPS72, WDR2, WDR14, WDR14, WDR36, WDR6, WDR7, WDR63, WDR64, WDR74, WDR74, WSB3, XYLT2, YARS, YARS, YLPM2, YPEL2, YWHAZ, YY2, ZCCHC12, ZCCHC15, ZEB2, ZFP15, ZMIZ3, ZNF13, ZNF134, ZNF175, ZNF233, ZNF239, ZNF278, ZNF301, ZNF359, ZNF359, ZNF409, ZNF411, ZNF424, ZNF512B, ZNF515, ZNF590, ZNF606, ZNF669, ZNF673, ZNF785.

Although the detection of a single marker can be sufficient to indicate a risk for colon cancer, it is preferred to use more than one marker, e.g. 2, 3, 4, 5, 6, 7, 8, 9, 10, 11, 12 or more markers in combination, especially if combined with statistical analysis. From a diagnostic point of view, a single autoantigen based diagnosis can be improved by increasing sensitivity and specificity by using a panel of markers where multiple autoantibodies are being detected simultaneously. Particular preferred combinations are of markers within one of the marker lists 2 to 31 as identified further herein.

The inventive markers are suitable protein antigens that are overexpressed in tumor and can be used to either identify cancerous tissue or to differentiate between specific stages of cancer and pre-cancerous development, or both. The markers usually cause an antibody reaction in a patient. Therefore the most convenient method to detect the presence of these markers in a patient is to detect antibodies against these marker proteins in a sample from the patient, especially a body fluid sample, such as blood or serum.

To detect an antibody in a sample it is possible to use marker proteins as binding agents and subsequently to detect bound antibodies. It is not necessary to use the entire marker proteins but it is sufficient to use antigenic fragments that are bound by the antibodies. “Antigenic fragment” herein relates to a fragment of the marker protein that causes an immune reaction against said marker protein in a human. Preferred antigenic fragments of any one of the inventive marker proteins are the fragments of the clones as identified by the UniqueID. Such antigenic fragments may be antigenic in a plurality of humans, such as at least 5, or at least 10 individuals.

“Diagnosis” for the purposes of this invention means the positive determination of colon carcinoma by means of the marker proteins according to the invention as well as the assignment of the patients to colon carcinoma. The term “diagnosis” covers medical diagnostics and examinations in this regard, in particular in-vitro diagnostics and laboratory diagnostics, likewise proteomics and peptide blotting. Further tests can be necessary to be sure and to exclude other diseases. The term “diagnosis” therefore likewise covers the differential diagnosis of colon carcinoma by means of the marker proteins according to the invention and the risk or prognosis of colon carcinoma.

Specific indications that can be identified with one or more of the inventive markers are cancer and pre-cancerous polyps, in particular also the differentiation between high-risk and low-risk polyps. Particular differentiations that can be made with the inventive markers and methods are distinguishing 1) healthy conditions vs. cancer plus pre-cancerous polyps (both high-risk and low-risk) 2) healthy conditions vs. cancer, 3) healthy conditions plus low-risk polyps vs. high-risk polyps plus cancer, 4) cancer vs. high-risk polyps vs. low-risk polyps vs. healthy conditions, 5) healthy conditions vs. low-risk polyps, 6) low-risk polyps vs. high-risk polyps and 7) high-risk polyps vs. cancer.

The invention can be used to distinguish 2 or more indications. If more than 2 indications, e.g. 3 or 4 such as in indication group 4) mentioned above, are distinguished it is preferred to use stepwise statistical analysis to distinguish the individual conditions. In preferred embodiments, in such a stepwise analysis, in a first step one indication is distinguished from the remaining indications, e.g. healthy conditions vs. the combined group of cancer, high-risk polyps and low-risk polyps. Stepwise, one additional indication is removed from the remaining group and distinguished from the next remaining indications. E.g. for group 4) it is preferred to distinguish in the second step low-risk polyps vs. the group of cancer and high-risk polyps, and consequently in the third step to distinguish cancer vs. high-risk polyps (see e.g. example 11). Such a statistical method is e.g. the binary tree analysis.

According to the invention, adenomatous villous, adenomatous tubulovillous, and co-occurrence of adenomatous tubular with tubulovillous polyps were classified as high-risk polyps while hyperplastic polyps and adenomatous tubular polyps were assigned to the low-risk group. The presence of high-risk polyps (and the presence of high-risk polyp markers) is an indication for a surgical intervention to remove the polyp since a rapid development to full cancer is likely. Contrary thereto, low-risk polyps do not require immediate surgical intervention but it is advised to further monitor the patients for the occurrence of high-risk polyp or cancer markers.

The invention thus also relates to a surgical method comprising detecting cancer or a high-risk polyp according to the present invention and removing said cancer or high-risk polyp. Of course, to be on the safe side it is also possible to similarly detect a low-risk polyp and remove said low-risk polyp.

In preferred methods of the invention the diagnosis of a risk for cancer comprises detecting polyps, in particular high-risk polyps and/or low-risk polyps, in a patient.

In particular the inventive method may comprise distinguishing between combined groups of cancer and pre-cancerous states, selected from distinguishing healthy conditions vs. cancer plus pre-cancerous polyps (both high-risk and low-risk), healthy conditions vs. cancer, healthy conditions plus low-risk polyps vs. high-risk polyps, healthy conditions plus low-risk polyps vs. high-risk polyps plus cancer, healthy conditions vs. low-risk polyps, low-risk polyps vs. high-risk polyps, and high-risk polyps vs. cancer. A positive result in distinguishing healthy conditions plus low-risk polyps vs. high-risk polyps plus cancer can prompt a further cancer test, in particular more invasive tests than a blood test such as an endoscopy or a biopsy.

The inventive markers are preferably grouped in sets of high distinctive value. Some sets excel at diagnosing or distinguishing 1, 2, 3, 4, 5, 6 or 7 of the above identified indications.

Preferred markers are of List 2, which comprise markers for all of the above indications 1) to 7).

List 2: Preferred marker protein set, suitable for multiple analytic distinctions; Proteins are identified by protein symbol: ACAA2, ACTL6B, ARHGEF1, ARHGEF10L, ASB13, ATXN2, C10orf76, C17orf28, TMEM98, C17orf90, AFP, AFP, COL3A1, CUL1, DCTPP1, DEF8, EIF4A2, RPA1, TACC2, ACTL6B, PLEKHO1, HAUS4, BTBD6, ISG15, LRRC4C, LTBP3, MACF1, MTCH2, NARFL, NBEAL2, KPNA2, PAPLN, PDIA3, PDP1, PI4KA, PKM2, PLAA, PLCG1, PLXND1, RNF40, RPL37A, SERPINA1, SLC4A3, SPRY1, TMCC2, TSTA3, UROD, PAICS, VPS18, ZNF410, ZNF668.

In particular embodiments, the invention provides the method of diagnosing colon cancer or the risk of colon cancer in a patient by detecting at least 2 of the marker proteins selected from the markers of List 2 in a patient comprising the step of detecting antibodies binding said marker proteins, detecting said marker proteins or antigenic fragments thereof in a sample of the patient. Also provided is a method of diagnosing colon cancer or the risk of colon cancer in a patient by detecting at least 20%, preferably at least 30%, especially preferred at least 40%, at least 50%, at least 60%, at least 70%, at least 80% at least 90% or all of the marker proteins selected from the markers of List 2 in a patient comprising the step of detecting antibodies binding said marker proteins, detecting said marker proteins or antigenic fragments thereof in a sample of the patient.

Further preferred marker sets according to the present invention are provided in example 7 as lists 3 to 31. Thus the present invention also provides the method of diagnosing colon cancer or the risk of colon cancer in a patient by detecting at least 2 of the marker proteins selected from the markers of List 3, 4, 5, 6, 7, 8, 9, 10, 11, 12, 13, 14, 15, 16, 17, 18, 19, 20, 21, 22, 23, 24, 25, 26, 27, 28, 29, 30, 31 or any combination thereof in a patient comprising the step of detecting antibodies binding said marker proteins, detecting said marker proteins or antigenic fragments thereof in a sample of the patient. Further provided is a method of diagnosing colon cancer or the risk of colon cancer in a patient by detecting at least 20%, preferably at least 30%, especially preferred at least 40%, at least 50%, at least 60%, at least 70%, at least 80% at least 90% or all, of the marker proteins selected from the markers of List 3, 4, 5, 6, 7, 8, 9, 10, 11, 12, 13, 14, 15, 16, 17, 18, 19, 20, 21, 22, 23, 24, 25, 26, 27, 28, 29, 30, 31 or any combination thereof in a patient comprising the step of detecting antibodies binding said marker proteins, detecting said marker proteins or antigenic fragments thereof in a sample of the patient.

Also provided is a method of diagnosing colon cancer or the risk of colon cancer in a patient by detecting a marker protein selected from any one of List 1 in a patient comprising the step of detecting antibodies binding said marker protein, detecting said marker protein or antigenic fragments thereof in a sample of the patient. Of course, preferably more than one marker protein is detected. As noted with regards to the marker combinations of sets of lists 2 to 31, preferably at least 2, but also 3, 4, 5, 6, 7, 8, 9, 10, 11, 12, 13, 14, 15, 16, 17, 18, 19, 20, 21, 22, 23, 24, or 25 or more, of the inventive marker proteins can be detected. This relates to any one of the inventive sets of lists 1 to 31. Even more preferred at least 20%, at least 30%, at least 40%, at least 50%, at least 60%, at least 70%, at least 80%, at least 90%, at least 95% or all of the markers of any set of any of the lists 1 to 31 are used in a diagnostic set. Such parts of at least 2 markers or at least 20% markers (or more as indicated) are also referred to as subsets herein.

Such a marker combination of a particular list or any combination of marker selection thereof are referred to herein as diagnostic set. Such sets constitute a further aspect of the invention and kits are provided comprising diagnostic agents (such as binding moieties) to detect such markers. The entire disclosure herein relates to both the inventive kits (that can be used in the inventive methods) as well as the methods themselves, that make use of agents that can be comprised in the kit.

Preferred combinations are of markers that are particularly indicative for a specific distinction as given in table 1 below.

Preferred marker combinations are of 2, 3, or 4 lists selected from lists 3, 4, 19 and 20. These lists, as well as any combination are particularly effective for distinguishing indication 1, healthy conditions vs. cancer plus pre-cancerous polyps (both high-risk and low-risk) and are preferably used therefore. Of course, not all of the markers are usually necessary since subsets also have sufficient diagnostic power. Preferably at least 2 markers or at least 20% of the markers (or any higher number as given above) of these lists or combined lists are used in the inventive methods.

Preferred marker combinations are of 2, 3, 4, 5 or 6 lists selected from lists 5, 6, 7, 8, 21 and 24. These lists, as well as any combination are particularly effective for distinguishing indication 2 healthy conditions vs. cancer and are preferably used therefore. Of course, not all of the markers are usually necessary since subsets also have sufficient diagnostic power. Preferably at least 2 markers or at least 20% of the markers (or any higher number as given above) of these lists or combined lists are used in the inventive methods.

Preferred marker combinations are of 2, 3, 4 or 5 lists selected from lists 9, 10, 11, 25 and 26. These lists, as well as any combination are particularly effective for distinguishing indication 3, healthy conditions plus low-risk polyps vs. high-risk polyps plus cancer, and are preferably used therefore. Of course, not all of the markers are usually necessary since subsets also have sufficient diagnostic power. Preferably at least 2 markers or at least 20% of the markers (or any higher number as given above) of these lists or combined lists are used in the inventive methods.

Preferred marker combinations are of 2 or 3 lists selected from lists 12, 13 and 27. These lists, as well as any combination are particularly effective for distinguishing indication 4 cancer vs. high-risk polyps vs. low-risk polyps vs. healthy conditions, and are preferably used therefore. Of course, not all of the markers are usually necessary since subsets also have sufficient diagnostic power. Preferably at least 2 markers or at least 20% of the markers (or any higher number as given above) of these lists or combined lists are used in the inventive methods.

A preferred marker combination is of the 2 lists selected from lists 14 and 28. These lists, as well as any combination are particularly effective for distinguishing indication 5, healthy conditions vs. low-risk polyps, and are preferably used therefore. Of course, not all of the markers are usually necessary since subsets also have sufficient diagnostic power. Preferably at least 2 markers or at least 20% of the markers (or any higher number as given above) of these lists or combined lists are used in the inventive methods.

Preferred marker combinations are of 2 or 3 lists selected from lists 15, 16 and 29. These lists, as well as any combination are particularly effective for distinguishing indication 6, low-risk polyps vs. high-risk polyps, and are preferably used therefore. Of course, not all of the markers are usually necessary since subsets also have sufficient diagnostic power. Preferably at least 2 markers or at least 20% of the markers (or any higher number as given above) of these lists or combined lists are used in the inventive methods.

Preferred marker combinations are of 2, 3 or 4 lists selected from lists 17, 18, 30 and 31. These lists, as well as any combination are particularly effective for distinguishing indication 7, high-risk polyps vs. cancer, and are preferably used therefore. Of course, not all of the markers are usually necessary since subsets also have sufficient diagnostic power. Preferably at least 2 markers or at least 20% of the markers (or any higher number as given above) of these lists or combined lists are used in the inventive methods.

In especially preferred embodiments, the combination is of lists 4 and 20, wherein the markers are selected from ACADVL, ADH5, AGFG1, ALDOA, ARHGAP21, ARHGEF1, ASB13, ATXN10, BCKDHA, BCS1L, BIN3, C10orf76, C17orf28, TMEM98, C17orf90, AFP, AFP, CCNI, CDK16, CHD8, COL3A1, CPLX1, CPNE6, CTDP1, CYC1, DCTPP1, DEF8, EID1, EIF4A2, RPA1, TACC2, ACTL6B, FAM160A2, HMGN2, FASN, C10orf2, HAUS4, JMJD4, BTBD6, IMP4, LAMB3, LCP1, LMO7, LMTK2, LRRC4C, LTBP3, MRPL47, MRPS11, MTCH2, MTSS1L, NARFL, NBEAL2, NME2, NRBP2, NOA1, PAPLN, PDIA3, PDP1, PI4KA, PIK3R2, PKM2, PLAA, PLCG1, PLXND1, PPP1R2, PPP4R1, PSMA2, PSMC2, RAD1, RBM4, RBM4, RPL26, RPL37A, SBF2, SERPINA1, SF3B4, SLC4A3, SLC25A29, SNRNP40, SPRY1, SSRP1, MGAT4B, TMUB2, ST3GAL3, TSTA3, UBE2L3, UMPS, UROD, USP7, PAICS, VASP, VPS18, VPS72, WDR13, YARS, ZNF410, ZNF668. These markers as well as the combined set of any one of at least 2 markers or at least 20% of said markers (or any higher number as indicated above) is particularly suitable for distinguishing healthy conditions vs. cancer plus pre-cancerous polyps (both high-risk and low-risk) and is preferably used for this diagnosis.

In especially preferred embodiments, the combination is of lists 5, 6, 7, 21, and 24 and wherein the markers are selected from A1BG, ACAA2, ACTL6B, ACTR1B, ADCK3, AFTPH, AKAP9, AP3D1, APBA3, ARAP1, CHST10, ARHGEF1, ARHGEF10L, ARHGEF17, ASB13, ASNA1, ATP5H, ATXN2, BCL6, C11orf80, TMEM98, C17orf90, AFP, AFP, C18orf32, C6orf136, CAMK1D, CCL28, CCND1, CDH2, CEP57, CKM, COL3A1, COL6A3, COL8A2, HSPA1A/HSPA1B, CPLX1, CSK, CSRNP1, CTDP1, CTNND2, CUL1, CUL9, CYC1, DCAF15, DCTPP1, DDX19B, DDX3Y, DDX54, DEF8, DENND5A, DNAJC10, DOCK10, DOCK9, DPYSL5, EEF1A1, EHMT2, EIF3L, EIF4A2, EML2, ERBB3, TACC2, ACTL6B, PLEKHO1, EXOSC8, FAM204A, FBN1, GEMIN2, GGA1, GP1BB, GPSM1, GSTP1, GTF3A, HADHA, HAUS4, HK1, HMG20B, HMGN2, HMGN2, HNRNPK, BTBD6, ICAM3, IFNGR2, IMP4, INTS7, ISG15, IVNS1ABP, JMJD7-PLA2G4B, KIAA0368, KIF3C, LIMD2, LPCAT1, LRRC4C, LRSAM1, MACF1, MAN2B1, MCM3AP, METTL2B, MTA1, MTCH2, MTSS1L, NAGLU, NARFL, NASP, NBEAL2, NCAN, NFKB2, NHEJ1, NME2, KPNA2, NPDC1, OTUD5, PAPLN, PBRM1, PBX2, PDP1, PDZD4, PHACTR3, LOC440354, PKD1L1, PKM2, PLAA, PLCG1, PLEKHA5, PLXNA1, PLXND1, PMVK, POGZ, POLN, POMGNT1, POTEE/POTEF, PPCDC, PPP4R1, PRDM2, PRKACA, PRKCSH, PRMT1, TSTA3, PRPF4B, PSMB4, PSMB6, QTRT1, RAD1, RBPJ, REV3L, RNF220, RPL24, RPL37A, RPL7, RPS7, RSL1D1, RSL1D1, RTN4, RUVBL2, SAFB2, SAMSN1, GRK6, SERBP1, SERPINA1, SETD1B, SGSH, SLC4A3, SLMAP, SNTA1, SPRY1, ST6GALNAC1, STAB1, SUOX, TAGLN3, TIAL1, TMC8, TMCO7, TNFAIP2, TPX2, TRAPPC3, ST3GAL3, TRIOBP, TRIP12, TRPC4AP, TSTA3, TTR, TTYH3, TUBB3, TUBGCP2, UBB, UMPS, UROD, UROD, PAICS, VPS18, YLPM1, YWHAZ, ZCCHC14, ZNF174, ZNF410, ZNF589. These markers as well as the combined set of any one of at least 2 markers or at least 20% of said markers (or any higher number as indicated above) is particularly suitable for distinguishing healthy conditions vs. cancer and is preferably used for this diagnosis.

In especially preferred embodiments, the combination is of lists 9-11 and 25-26 and wherein the markers are selected from ABCE1, ACAA2, ACTL6B, ADH5, AKT2, ANKRD36B, ANXA6, APBB1IP, ARHGAP21, ARHGEF10L, ARHGEF25, ASAP1, ASB13, ASPSCR1, AXIN1, BCKDHA, C10orf76, TMEM98, C17orf90, AFP, AFP, C18orf32, PECAM1, C6orf136, CCL28, IK, CDK16, CDT1, CERCAM, CHCHD8, CLDN5, CLU, COMMD9, COX7A2L, CPE, CPNE2, DBI, DCAF11, DCTPP1, DDR1, DDX27, DENR, DHX36, DHX58, DLG5, DTNBP1, EID1, EIF4A2, EPC1, TACC2, ACTL6B, PLEKHO1, EXOSC4, FAM213A, FBLL1, FBRS, FKBP15, FLII, FN1, GNAO1, GOSR1, GRK6, GTF2B, HAPLN3, HARS, HAUS4, HDAC6, HEBP2, HLA-C, JMJD4, HNRNPA2B1, HNRNPK, HNRNPR, HNRPLL, ELAC2, HSPA8, INPP5K, INPPL1, INTS1, INTS1, ISG15, KDM4A, LAMB1, LCP1, LOC100132116, LRIG1, LRIG1, LRRC16B, LRRC4C, LTBP3, LYSMD2, MAF1, MRPL38, MTCH1, MTCH2, RPL17, MYO1C, MYO5A, NARF, NARFL, NBEAL2, NBR1, NDUFB10, NME2, KPNA2, OTUD1, PABPC1, PAPLN, PDIA3, LOC440354, PI4KA, PIK3CD, PIK3IP1, PJA1, PKD1L1, PKM2, PLAA, PKP3, PLXNA3, PLXND1, POMT1, PPP1R13B, PPP4R1, PPP5C, PRDX5, PRRT1, PRRT2, PSKH1, PSMB4, REC8, RGS19, RHBDD2, RPL4, RNF40, RPL13, RPL17, RPL22, RPL24, RPL37A, RPN1, SAT2, SERPINA1, SERPINH1, SGSH, SH3BP2, SLC25A20, SMCHD1, SNTA1, SPRY1, SRRM2, STMN4, TBC1D9B, TBCB, TIAL1, TIAM2, TMC8, TMCC2, TPM3, ZWINT, TRAPPC3, TRAPPC4, TSTA3, TTYH1, GPX4, TUBB3, UBC, UBE2N, USP48, VARS, PAICS, VPS18, YLPM1, YPEL1, ZCCHC11, ZFP14, ZNF133, ZNF232, ZNF410, ZNF668, ZNF672. These markers as well as the combined set of any one of at least 2 markers or at least 20% of said markers (or any higher number as indicated above) is particularly suitable for distinguishing healthy conditions plus low-risk polyps vs. high-risk polyps plus cancer and is preferably used for this diagnosis.

In especially preferred embodiments, the combination is of lists 14 and 28 and wherein the markers are selected from AHSG, AKR1B1, AP1B1, ARPP21, ATP2A3, BMS1P5, BTBD11, CANX, CCL28, CDK16, CPNE1, DCTPP1, DDX19A, DHX8, EHD4, RPA1, ELAVL1, TACC2, FBXO21, C10orf2, HMGCL, IKBKAP, ITGAL, LMTK2, LRPAP1, MAGED1, MAPKAP1, MARS, MAST2, MATR3, MUC2, MYO5A, NAV2, NDUFS2, NEFL, PDP1, PEX19, PFKL, PKM2, PLAA, PLEKHM2, MED4 (includes EG:29079), PNCK, PPP2R5C, PSMD6, PTPRS, BAD, RNF10, RPL17, RPL37A, ACO2 (includes EG:11429), SLC4A3, STK17A, SUV420H1, TBC1D7, TBC1D9B, TRMT2A, TUBA1B, TUBB6, WDR1, WDR5, ZNF277, ZNF514. These markers as well as the combined set of any one of at least 2 markers or at least 20% of said markers (or any higher number as indicated above) is particularly suitable for distinguishing healthy conditions vs. low-risk polyps and are preferably used for this diagnosis.

Some markers are more preferred than others. Especially preferred markers are those which are represented at least 5, at least 6, at least 7, at least 8, at least 9, at least 10, at least 11, at least 12 times in any one of lists 3 to 31. These markers are preferably used in any one of the inventive methods or sets.

Less preferred markers are selected from AGBL5, AKT1, AKT2, C21orf2, C6orf192, C9orf43, COASY, EFNA3, EPRS, FAS, GOLGA4, GOLGB1, HDAC5, HIP1R, HSPA4, KIAA1416, CHD7, KIF2C, LMNA, MAPKAPK3, MBD2, NFYA, NHSL1, NUCB1, NY-CO-16, RPS6KA1, RPS6KA2, SDCCAG1, SDCCAG3, SREBF2, STAU1, STUB1, TAX1BP1, TRIP4, TTLL7, WBP2, Znf292, BHMT2, BMX, Cbx5, CEA, C-myc, CSNK1G2, CTAG1A, DAPK1, FGFR4, GRK7, HDAC1, Her-2/neu, HMGN2, HMMR, HSPH1, IGLC1, Imp1, IRAK4, ITGA6, KDR, Koc, LGR6, LIMS1, LMTK2, MAGEA3, MAPKAPK5, MFAP2, MKNK1, NAP1L1, NEK3, NMDAR, NOXA1, NY-CO-41, NY-CO-8, NY-ESO-1, P62, PBK, PDE4A, PDGFRB, PDXK, PFDN5, PIM1, PKN1, PKN2, PRKCD, RBMS1, RBPJ, RIOK1, SALL2, SCP2, SDCCAG10, SDCCAG8, Seb4D, SRC, SSRP1, SSX2, STARD10, STK4, TAF10, TDRD6, TP53, TPM4, TRIM21, TSHZ1, TSLP, UBE3A, USH1C, ZNF706. Preferably none of these markers is used in the inventive methods or present in one of the inventive set. As an alternative to this exclusion, these less preferred markers may just not be a requirement of any of the inventive sets, i.e. the sets or lists herein may be read as if any one of these less preferred markers is not a recited therein.

The present invention also relates to a method of selecting such at least 2 markers (or more as given above) or at least 20% of the markers (or more as given above) of any one of the inventive sets with high specificity. Such a method includes comparisons of signal data for the inventive markers of any one of the inventive markers sets, especially as listed in lists 1 to 31, with said signal data being obtained from controls samples of known conditions or indications and further statistically comparing said signal data with said conditions thereby obtaining a significant pattern of signal data capable of distinguishing the conditions of the known control samples.

In particular, the controls may comprise one or more cancerous control (preferably at least 5, or at least 10 cancerous controls) and/or a pre-cancerous polyp (e.g. high risk or low risk polyps) control (preferably at least 5, or at least 10 pre-cancerous controls) and/or a healthy control (preferably at least 5, or at least 10 healthy controls). Preferably at least 2 different indications are selected that shall be distinguished. In preferred embodiments, the control comprises samples for the indications selected from indications 1), 2), 3), 4), 5), 6), and 7) as mentioned above.

The controls can be used to obtain a marker dependent signal pattern as indication classifier. Such a signal pattern can be obtained by routine statistical methods, such as binary tree methods. Common statistical methods calculate a (optionally multi-dimensional) vector within the multitude of control data signal values as diagnostically significant distinguishing parameter that can be used to distinguish one or more indications from other one or more indications. The step usually comprises the step of “training” a computer software with said control data. Such pre-obtained training data or signal data can be provided on a computer-readable medium to a practitioner who performs the inventive diagnosis.

Preferably, the method comprises optimizing the selection process, e.g. by selecting alternative or additional markers and repeating said comparison with the controls signals, until a specificity and/or sensitivity of at least 75% is obtained, preferably of at least 80%, at least 85%, at least 90%, at least 95%.

Thus as mentioned, the present invention also relates to diagnosing cancer or pre-cancerous polyps, preferably wherein said precancerous polyps are distinguished between high-risk polyps and low-risk polyps, wherein said high-risk polyps comprise adenomatous villous, adenomatous tubulovillous, or co-occurrence of adenomatous tubular with tubulovillous polyps. For any one of the particular distinctions, preferably at least one of the markers or marker lists or subsets thereof, which contains said indication selected from cancer, high-risk polyps, low risk polyps as given above and in table 1 is used.

“Marker” or “marker proteins” are diagnostic indicators found in a patient and are detected, directly or indirectly by the inventive methods. Indirect detection is preferred. In particular, all of the inventive markers have been shown to cause the production of (auto)antigens in cancer patients or patients with a risk of developing cancer. The easiest way to detect these markers is thus to detect these (auto)antibodies in a blood or serum sample from the patient. Such antibodies can be detected by binding to their respective antigen in an assay. Such antigens are in particular the marker proteins themselves or antigenic fragments thereof. Suitable methods exist in the art to specifically detect such antibody-antigen reactions and can be used according to the invention. Preferably the entire antibody content of the sample is normalized (e.g. diluted to a preset concentration) and applied to the antigens. Preferably the IgG, IgM, IgD, IgA or IgE antibody fraction, is exclusively used. Preferred antibodies are IgG. Preferably the subject is a human.

Binding events can be detected as known in the art, e.g. by using labeled secondary antibodies. Such labels can be enzymatic, fluorescent, radioactive or a nucleic acid sequence tag. Such labels can also be provided on the binding means, e.g. the antigens as described in the previous paragraph. Nucleic acid sequence tags are especially preferred labels since they can be used as sequence code that not only leads to quantitative information but also to a qualitative identification of the detection means (e.g. antibody with certain specificity). Nucleic acid sequence tags can be used in known methods such as Immuno-PCR. In multiplex assays, usually qualitative information is tied to a specific location, e.g. spot on a microarray. With qualitative information provided in the label, it is not necessary to use such localized immunoassays. In is possible to perform the binding reaction of the analyte and the detection means, e.g. the serum antibody and the labeled antigen, independent of any solid supports in solution and obtain the sequence information of the detection means bound to its analyte. A binding reaction allows amplification of the nucleic acid label in a detection reaction, followed by determination of the nucleic acid sequence determination. With said determined sequence the type of detection means can be determined and hence the marker (analyte, e.g. serum antibody with tumor associated antigen specificity).

In preferred embodiments of the invention the step of detecting antibodies binding said marker proteins, detecting said marker proteins or antigenic fragments thereof comprises comparing said detection signal with detection signals of a healthy control and comparing said detection signals, wherein an increase in the detection signal indicates colon cancer or said risk of colon cancer.

In preferred embodiments of the invention the step of detecting antibodies binding said marker proteins, detecting said marker proteins or antigenic fragments thereof comprises comparing said detection signal with detection signals of a cancerous control or a pre-cancerous polyp (e.g. high risk or low risk polyps) control and comparing said detection signals. Alternatively or in addition, the comparison can also be with a healthy control, a control of a low-risk polyp, a control with a high risk polyp or any combination thereof. In preferred embodiments, the control comprises the indications that are intended to be distinguished, such as indications 1), 2), 3), 4), 5), 6), and 7) as mentioned above. In particular preferred, especially in cases of using more marker sets of 2 or more markers as mentioned above, a statistical analysis of the control is performed, wherein the controls are used to obtain a marker dependent signal pattern as indication classifier and the marker dependent signals of the sample to be analysed is compared with and/or fitted onto said pattern thereby obtaining information of the diagnosed condition or indication. Such a signal pattern can be obtained by routine statistical methods, such as binary tree methods. Common statistical methods calculate a (optionally multi-dimensional) vector within the multitude of control data signal values as diagnostically significant distinguishing parameter that can be used to distinguish one or more indications from other one or more indications. Such statistical analysis is usually dependent on the used analytical platform that was used to obtain the signal data, given that signal data may vary from platform to platform. Such platforms are e.g. different microarray or solution based setups (with different labels or analytes—such as antigen fragments—for a particular marker). Thus the statistical method can be used to calibrate each platform to obtain diagnostic information with high sensitivity and specificity. The step usually comprises the step of “training” a computer software with said control data. Alternatively, pre-obtained training data can be used. Such pre-obtained training data or signal data can be provided on a computer-readable medium to a practitioner.

In further embodiments a detection signal from the sample of a patient in amplitude of at least 60%, preferably at least 80%, of the cancerous control indicates colon cancer or said risk of colon cancer.

Usually not all of the inventive markers or detection agents may lead to a signal. Nevertheless only a fraction of the signals is suitable to arrive at a diagnostic decision. In preferred embodiments of the invention a detection signal in at least 60%, preferably at least 70%, least 75%, at least 85%, or in particular preferred at least 95%, even more preferred all, of the used markers indicates colon cancer or said risk of colon cancer.

The present diagnostic methods further provide necessary therapeutic information to decide on a surgical intervention. Therefore the present invention also provides a method of treating a patient comprising colon cancer or having a polyp with a high risk of developing colon cancer, comprising detecting cancer or a polyp with a high risk of developing colon cancer according to any aspect or embodiment of the invention and removing said colon cancer or polyp. “Stratification or therapy control” for the purposes of this invention means that the method according to the invention renders possible decisions for the treatment and therapy of the patient, whether it is the hospitalization of the patient, the use, effect and/or dosage of one or more drugs, a therapeutic measure or the monitoring of a course of the disease and the course of therapy or etiology or classification of a disease, e.g., into a new or existing subtype or the differentiation of diseases and the patients thereof. In a further embodiment of the invention, the term “stratification” covers in particular the risk stratification with the prognosis of an outcome of a negative health event.

One skilled in the art is familiar with expression libraries, they can be produced according to standard works, such as Sambrook et al, “Molecular Cloning, A laboratory handbook, 2nd edition (1989), CSH press, Cold Spring Harbor, N.Y. Expression libraries are also preferred which are tissue-specific (e.g., human tissue, in particular human organs). Members of such libraries can be used as inventive antigen for use as detection agent to bind analyte antibodies. Furthermore included according to the invention are expression libraries that can be obtained by exon-trapping. A synonym for expression library is expression bank. Also preferred are protein biochips or corresponding expression libraries that do not exhibit any redundancy (so-called: Uniclone® library) and that may be produced, for example, according to the teachings of WO 99/57311 and WO 99/57312. These preferred Uniclone libraries have a high portion of non-defective fully expressed proteins of a cDNA expression library. Within the context of this invention, the antigens can be obtained from organisms that can also be, but need not be limited to, transformed bacteria, recombinant phages, or transformed cells from mammals, insects, fungi, yeasts, or plants. The marker antigens can be fixed, spotted, or immobilized on a solid support. Alternatively, is also possible to perform an assay in solution, such as an Immuno-PCR assay.

In a further aspect, the present invention provides a kit of diagnostic agents suitable to detect any marker or marker combination as described above, preferably wherein said diagnostic agents comprise marker proteins or antigenic fragments thereof suitable to bind antibodies in a sample, especially preferred wherein said diagnostic agents are immobilized on a solid support or in solution, especially when said markers are each labelled with a unique label, such as a unique nucleic acid sequence tag. The inventive kit may further comprise detection agents, such as secondary antibodies, in particular anti-human antibodies, and optionally also buffers and dilution reagents. The invention therefore likewise relates to the object of providing a diagnostic device or an assay, in particular a protein biochip, ELISA or Immuno-PCR assay, which permits a diagnosis or examination for colon carcinoma.

Additionally, the marker proteins (as binding moieties for antibody detection) can be present in the respective form of a fusion protein, which contains, for example, at least one affinity epitope or tag. The tag may be one such as contains c-myc, his tag, arg tag, FLAG, alkaline phosphatase, VS tag, T7 tag or strep tag, HAT tag, NusA, S tag, SBP tag, thioredoxin, DsbA, a fusion protein, preferably a cellulose-binding domain, green fluorescent protein, maltose-binding protein, calmodulin-binding protein, glutathione S-transferase, or lacZ, a nanoparticle or a nucleic acid sequence tag. Such a nucleic acid sequence can be e.g. DNA or RNA, preferably DNA.

In all of the embodiments, the term “solid support” covers embodiments such as a filter, a membrane, a magnetic or fluorophore-labeled bead, a silica wafer, glass, metal, ceramics, plastics, a chip, a target for mass spectrometry, or a matrix. However, a filter is preferred according to the invention.

As a filter, furthermore PVDF, nitrocellulose, or nylon is preferred (e.g., Immobilon P Millipore, Protran Whatman, Hybond N+ Amersham).

In another preferred embodiment of the arrangement according to the invention, the arrangement corresponds to a grid with the dimensions of a microtiter plate (8-12 wells strips, 96 wells, 384 wells, or more), a silica wafer, a chip, a target for mass spectrometry, or a matrix.

Preferably the inventive kit also comprises non-diagnostic control proteins, which can be used for signal normalization. These control proteins bind to moieties, e.g. proteins or antibodies, in the sample of a diseased patient same as in a healthy control. In addition to the inventive marker proteins any number, but preferably at least 2 controls can be used in the method or in the kit.

Preferably the inventive kit is limited to a particular size. According to these embodiments of the invention the kit comprises at most 3000 diagnostic agents, preferably at most 2500 diagnostic agents, at most 2000 diagnostic agents, at most 1500 diagnostic agents, at most 1200 diagnostic agents, at most 1000 diagnostic agents, at most 800 diagnostic agents, at most 500 diagnostic agents, at most 300 diagnostic agents, at most 200 diagnostic agents, at most 100 diagnostic agents, such as marker proteins or antigenic fragments thereof.

In especially preferred embodiments of the invention the kit further comprises a computer-readable medium or a computer program product, such as a computer readable memory devices like a flash storage, CD-, DVD- or BR-disc or a hard drive, comprising signal data for the control samples with known conditions selected from cancer or a pre-cancerous polyp (such as high risk polyps and/or low risk polyps) and/or of healthy controls, and/or calibration or training data for analysing said markers provided in the kit for diagnosing colon cancer or distinguishing conditions or indications selected from healthy conditions, cancer, high-risk polyps and low-risk polyps. Especially preferred are the indications 1), 2), 3), 4), 5), 6) and 7) mentioned above.

The kit may also comprise normalization standards, that result in a signal independent of a healthy condition and cancerous or pre-cancerous condition. Such normalization standards can be used to obtain background signals. Such standards may be specific for ubiquitous antibodies found in a human, such as antibodies against common bacteria such as E. coli. Preferably the normalization standards include positive and negative (leading to no specific signal) normalization standards.

The present invention is further illustrated by the following figures and examples, without being limited to these embodiments of the invention.

### FIGURES

FIG. 1: Example of a scanned 16k protein-microarray. In the detail right side, subarray 6 is depicted.

FIG. 2: Subsets of classifier markers form the panels defined by the 50 classifier markers for distinguishing Cancer vs. Controls were selected and randomly chosen subsets of 2-49 markers (x-axis) were selected 1000-times. The classification success obtained by that subset using the nearest centroid classifier algorithm is shown on the y-axis.

FIG. 3: The 80 classifier markers for distinguishing Disease (Cancer & polyps) vs. Controls were selected and randomly chosen subsets of 2-79 markers (x-axis) were selected 1000-times. The classification success obtained by that subset using the nearest centroid classifier algorithm is shown on the y-axis.

### EXAMPLES

### Example 1

### Patient Samples

Biomarker screening has been performed with serum samples from a test set of serum samples derived from 49 individuals with confirmed colon-carcinoma, 50 healthy controls, 17 patients with high-risk polyps, and 18 patients with low-risk polyps (n=134). All these individuals have been elucidated by a positive FOBT test and underwent colonoscopy. The differentiation of carcinoma (denoted Carc), high and low risk polyps (denoted HR and LR, respectively) and controls (denoted Contr) was conducted during clinical examination of patients and tissue samples.

### Example 2

### Immunoglobuline (IgG) Purification from the Serum or Plasma Samples

The patient serum or plasma samples were stored at −80° C. before they were put on ice to thaw them for IgG purification using Melon Gel 96-well Spin Plate according the manufacturer's instructions (Pierce). In short, 10 μl of thawed sample was diluted in 90 μl of the equilibrated purification buffer on ice, then transferred onto Melon Gel support and incubated on a plate shaker at 500 rpm for 5 minutes. Centrifugation at 1,000×g for 2 minutes was done to collect the purified IgG into the collection plate.

Protein concentrations of the collected IgG samples were measured by absorbance measures at 280 nm using an Epoch Micro-Volume Spectrophotometer System (Biotec, USA). IgG-concentrations of all samples were concentration-adjusted and 0.6 mg/ml of samples were diluted 1:1 in PBS2× buffer with TritonX 0.2% and 6% skim milk powder for microarray analyses.

### Example 3

### Microarray Design

A protein-chip named “16k protein chip” from 15284 human cDNA expression clones derived from the Unipex cDNA expression library plus technical controls was generated. Using this 16k protein chip candidate markers were used to identify autoantibody profiles suitable for unequivocal distinction of healthy, malign and benign colon tumors.

Protein-microarray generation and processing was using the Unipex cDNA expression library for recombinant protein expression in E. coli. His-tagged recombinant proteins were purified using Ni-metal chelate chromatography and proteins were spotted in duplicates for generation of the microarray using ARChipEpoxy slides.

### Example 4

### Preparation, Processing and Analyses of Protein Microarrays

The microarray with printed duplicates of the protein marker candidates was blocked with DIG Easy Hyb (Roche) in a stirred glass tank for 30 minutes. Blocked slides were washed 3× for 5 minutes with fresh PBSTritonX 0.1% washing buffer with agitation. The slides were rinsed in distilled water for 15 seconds to complete the washing step and remove leftovers from the washing buffer. Arrays were spun dry at 900 rpm for 2 minutes. Microarrays were processed using the Agilent Microarray Hybridisation Chambers (Agilent) and Agilent's gasket slides filled with 490 μl of the prepared sample mixture and processed in a hybridization oven for 4 h at RT with a rotation speed of 12. During this hybridization time the samples were kept under permanent rotating conditions to assure a homolog dispensation.

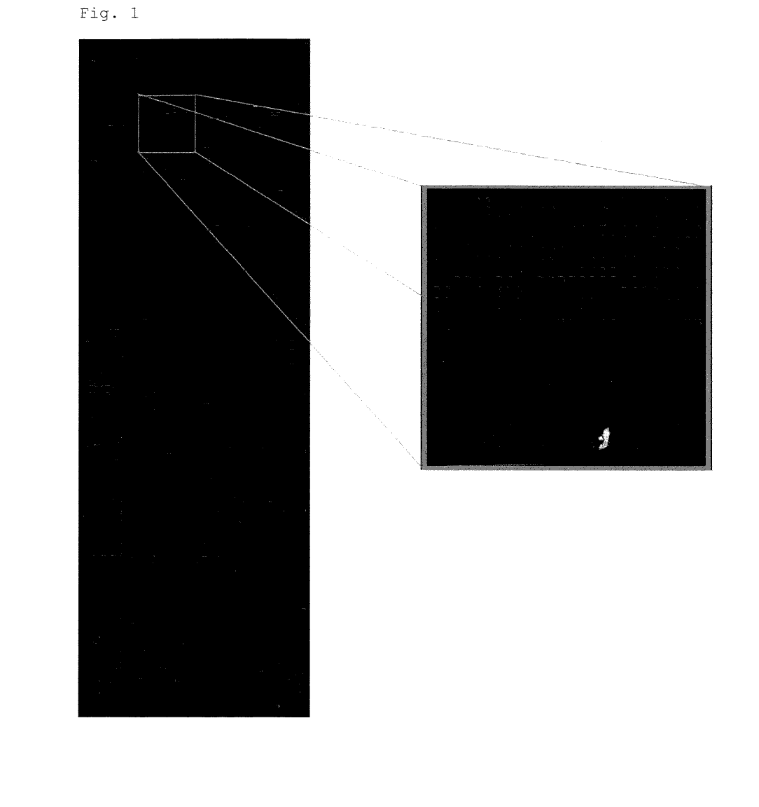

After the hybridization was done, the microarray slides were washed 3× with the PBSTritonX 0.1% washing buffer in the glass tank with agitation for 5 minutes and rinsed in distilled water for about 15 seconds. Then, slides were dried by centrifugation at 900 rpm for 2 minutes. IgG bound onto the features of the protein-microarrays were detected by incubation with cy5 conjugated Alexa Fluor® 647 Goat Anti-Human IgG (H+L) (Invitrogen, Lofer, Austria), diluted in 1:10,000 in PBSTritonX 0.1% and 3% skim milk powder using rotating conditions for 1 h, with a final washing step as outlined above. Microarrays were then scanned and fluorescent data extracted from images (FIG. 1) using the GenePixPro 6.0 software (AXON).

### Example 5

### Data Analysis

Data were 1) quantil normalised and alternatively 2) normalised in DWD transformation for removal of batch effects, when samples were processed on microarrays in 4 different runs; data analyses was conducted using BRB array tools (web at linus.nci.nih.gov/BRB-ArrayTools.html) upon the 2 different normalization strategies (quantil and DWD normalized).

For identification of tumor marker profiles and classifier markers, class prediction analyses applying leave-one-out cross-validation was used. Classifiers were built for distinguishing each of the four classes of samples denoted “Carc” carcinoma patients, “HR” patients harboring high-risk polyps, “LR” patients harboring low-risk polyps, and “Contr” individuals with no carcinoma or polyps. In addition different combinations of classes were also built as listed in the table below (Tab. 1) and again class prediction analysis was conducted for differentiation of these different combinations.

TABLE 1
Colon tumor marker Classifiers defined for separation of different
indications (Carc.: Colon Carcinoma, Contr.: controls with
no colon or polyp/tumor; LR/HR: low/high risk polyps;
vs . . . versus). The various examples upon data analyses after
different normalization strategies A) and B) are given.

| | A) examples | B) examples |
|---|---|---|
| | with quantil | with DWD |
| Contrast analysed | normalisation | normalisation |
| 1) Contr. vs. Carc & HR & LR | 7.1, 7.2 | 7.17, 7.18 |
| 2) Contr. vs. Carc. | 7.3-7.6 | 7.19-7.22 |
| 3) Contr. & LR vs. Carc. & HR | 7.7-7.9 | 7.23, 7.24 |
| 4) Carc. vs. Hr vs. LR vs. Contr. | 7.10, 7.11 | 7.25 |
| 5) Contr. vs. LR | 7.12 | 7.26 |
| 6) LR vs. HR | 7.13, 7.14 | 7.27 |
| 7) HR vs. Carc. | 7.15, 7.16 | 7.28, 7.29 |

### Example 6

### Results Summary

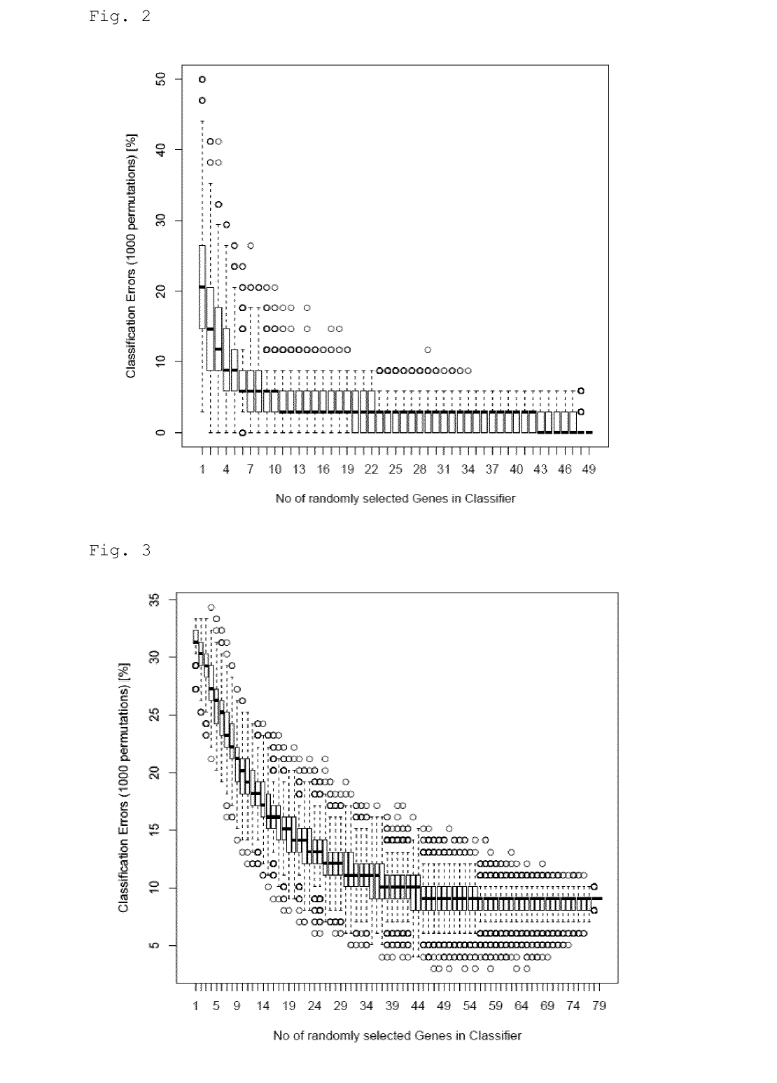

For distinguishing 1) Contr vs “Carc-HR-LR”, 2) Controls vs Carcinomas, or 3) Contr_LR vs Carc_HR, 47 genes were present in at least 5 classifier lists. The classification success with respect to different contrasts (differentiation of different patient classes and combinations thereof) and presence of 47 preferred List 2 markers is given in Table 3. As shown, the number of markers out of the 47 selected. It was also shown that using only isolated or only 2 markers from the present classifier sets enables correct classification of >60% (Example 8, FIGS. 2 & 3). Therefore the marker-lists, subsets and single markers (antigens; proteins; peptides) are of particular diagnostic values.

In addition it has already been shown that peptides deduced from proteins or seroreactive antigens can be used for diagnostics and in the published setting even improve classification success (Syed 2012; Journal of Molecular Biochemistry; Vol 1, No 2, www.jmolbiochem.com/index.php/JmolBiochem/article/view/54).

TABLE 2
Data upon Quantil and DWD normalisation have been analyzed with respect to different
Contrasts given and classifiers generated by different class prediction methods, - the
numbers of classifiers is depicted; out of these a valuable number of preferred 47 List
2 markers is present within those classifiers lists. Correct classification for each
example is given in %. The right column refers to the number of the example.

| | | number of | | | | |
|---|---|---|---|---|---|---|
| | number of | markers | distinguished | | correct | |
| normalisation | markers | within list 2 | contrast | contrast # | classification | Example |
| Quantil | 261 | 20 | 1) Contr vs | 1 | 81% | 7.1 |
| normalised | 50 | 23 | “Carc-HR-LR” | 1 | 78% | 7.2 |
| data | 50 | 5 | 2) Controls vs | 2 | 97% | 7.3 |
| | 50 | 2 | Carcinomas | 2 | 80% | 7.4 |
| | 50 | 29 | | 2 | 79% | 7.5 |
| | 216 | 47 | | 2 | 79% | 7.6 |
| | 226 | 34 | 3) Contr_LR vs | 3 | 83% | 7.7 |
| | 50 | 22 | Carc_HR | 3 | 80% | 7.8 |
| | 40 | 8 | | 3 | 82% | 7.9 |
| | 447 | 26 | 4) Carc vs Hr vs | 4 | 85% | 7.10 |
| | 175 | 36 | LR vs Contr | 4 | 93% | 7.11 |
| | 40 | 3 | 5) Contr vs LR | 5 | 75% | 7.12 |
| | 50 | 1 | 6) LR vs HR | 6 | 80% | 7.13 |
| | 38 | 1 | | 6 | 83% | 7.14 |
| DWD | 50 | 29 | 1) Contr vs | 1 | 83% | 7.17 |
| normalised | 80 | 36 | “Carc-HR-LR” | 1 | 83% | 7.18 |
| data | 50 | 16 | 2) Controls vs | 2 | 94% | 7.19 |
| | 50 | 30 | Carcinomas | 2 | 87% | 7.20 |
| | 30 | 23 | | 2 | 89% | 7.21 |
| | 40 | 7 | | 2 | 83% | 7.22 |
| | 143 | 25 | 3) Contr LR | 3 | 76% | 7.23 |
| | 50 | 24 | vs Carc_HR | 3 | 81% | 7.24 |
| | 437 | 27 | 4) Carc vs Hr vs | 4 | 93% | 7.25 |
| | | | LR vs Contr | | | |
| | 50 | 4 | 5) Contr vs LR | 5 | 89% | 7.26 |
| | 40 | 7 | 7) HR vs Carc | 7 | 68% | 7.28 |

Different classifier lists have been elucidated for the “contrasts” listed in Table 1,—upon A) quantil normalization (QNORM) and B) DWD transformation.

Classifier markers (n=1150) were identified according to List 1. 441 markers were present in in both sets of A) and B) normalized data; 431 markers were present in classifier-sets upon A) quantil normalization, and 270 are present in classifiers upon DWD transformation.

Upon marker annotation 225 markers present an identical protein, 225 duplicates can be removed and remaining unique make up a list of 959 single Unigenes; thereof 374 are present in both sets of QNORM (A) and DWD (B) normalized data; 320 are present in classifiers upon QNORM, and an additional 265 are present in classifiers upon DWD transformation. Taken together based on the Unigene ID—3 different markers are present in 17 classifiers, 2 markers in 16 classifiers, 7 markers in 14 classifiers etc.

### Example 7

### Detailed Results

### Quantil-Normalised Data

### Example 7.1

### Contr Vs “Carc-HR-LR”—“Grid of Alpha Levels” & 1.25 Fold

The following markers were identified according to this example:

List 3: ACADVL, ACTL6B, ADAP1, ADCY1, ADD2, AK1, AKR1C4, ANGPTL2, ANXA6, APBB1, APBB1, ARHGEF1, ARL16, GDAP1L1, ASB13, ATP6V1H, ATXN10, BCS1L, BIN3, C10orf76, C16orf58, C16orf58, C17orf28, TMEM98, C17orf90, AFP, AFP, CALR, CALR, CAMK1D, CAPN2, CCDC64, CCDC88B, IK, CDH23, CDK16, CDK9, CHID1, CHID1, CKM, CLUAP1, COA5, COL6A3, CPNE9, CTAGE5, CUL1, CUX1, DBR1 (includes EG:323746), DDX19B, DDX41, DHX58, DNAH1, DOCK9, DOCK9, DPP3, DPYSL3, DRAP1, DRG2, EHMT2, EIF3E, EIF4A2, RPA1, EPS8L2, TACC2, EXOC6B, EXTL1, FAM108C1, FAM214A, HMGN2, FASN, FBXO44, FKBP15, FLII, FLNA, FLNA, FNTA, GBAS, GNPTG, GPCPD1, GPSM1, GRK6, GSS, HAUS4, HMGA1, HMGB2, HNRPDL, HSP90B1, HSPA8, SNAPIN, IDH3G, IDS, IFITM1, IFRD1, INA, IVNS1ABP, JARID2, KCNAB2, KCNG1, KIAA0368, KIAA1598, KPNA2, KPNB1, LAMB1, LGALS3, LMO4, LOC494127, HMGB2, LPCAT1, LPPR3, LRP5, LRRN2, LSM12 (includes EG:124801), LTBP3, MACF1, MED19, MICAL1, MRPL28 (includes EG:10573), MRPL38 (includes EG:303685), MTCH2, MYCBP2, NAPA (includes EG:108124), NARF, NELL2, NISCH, NMT2, NOA1, KPNA2, NRBP2, NOA1, NT5C, NUMA1, OAS3, OCRL, OGT, PAICS, PDDC1, PDIA4, PDZD4, PEA15, PEX5, PHF2, PHF8, PI4KA, PITPNC1, PKM2, PLAA, PKM2, PLAA, PLD2, PLXND1, POLN, PPIF, PPP1CA, PPP1R2, PPP4C, PSMC3, PSMD2, PSMD2, PTPN5, PTPN6, RAN, RIPK1, RNH1, RPL10A, RPL17, RPL24, RPL26, RPL26, RPN1, RRM1, RSL1D1, RUVBL2, SAT1, SCHIP1, SERPINB1, SFTPA1, SLC4A3, SKIV2L, SLUT (includes EG:10569), SMURF2, SNRNP200, SNX1, SORL1, SPAG17, SPECC1L, SPP1 (includes EG:20750), STK25, TBCD, TBL3, TCERG1, TCF3, HSPA5, TFAP4, THAP7, VIM, TKT, TMCC2, TMEM120A, TMSB10/TMSB4X, TPT1 (includes EG:100043703), TSC22D3, TUBA1B, TUBA1B, TUBB4A, TWF2, TYK2, UROD, PAICS, VAT1, VPS13C, VTI1B, WDR18, WDR35, YME1L1, YWHAQ, ZC3H10, ZNF12, ZNF354B, ZSWIM4.

Using microarray data for distinguishing “Contr” versus “Carcinoma & polyps” class prediction analysis was performed using feature selection upon “optimization of the grid of alpha levels” and “1.25 fold change of median intensities between classes”. The 1-Nearest Neighbour Predictor gave the best correct classification of 81% using 216 markers.

Genes significantly different between the classes at the 0.01, 0.005, 0.001 and 0.0005 significance levels were used to build four predictors. The predictor with the lowest cross-validation mis-classification rate was selected. The best compound covariate classifier consisted of genes significantly different between the classes at the 5e-04 significance level. The best diagonal linear discriminant analysis classifier consisted of genes significantly different between the classes at the 5e-04 significance level. The best 1-nearest neighbor classifier consisted of genes significantly different between the classes at the 0.001 significance level. The best 3-nearest neighbors classifier consisted of genes significantly different between the classes at the 5e-04 significance level. The best nearest centroid classifier consisted of genes significantly different between the classes at the 5e-04 significance level. The best support vector machines classifier consisted of genes significantly different between the classes at the 0.01 significance level. The best Bayesian compound covariate classifier consisted of genes significantly different between the classes at the 5e-04 significance level. Only genes with the fold-difference between the two classes exceeding 1.25 were used for class prediction.

Repeated 1 times K-fold (K=20) cross-validation method was used to compute mis-classification rate.

Performance of classifiers during cross-validation.

| | | | | | | | | |
|---|---|---|---|---|---|---|---|---|
| | | | Diagonal | | | | | Bayesian |
| | | Compound | Linear | | | | Support | Compound |
| | | Covariate | Discriminant | | 3-Nearest | Nearest | Vector | Covariate |
| Array | Class | Predictor | Analysis | 1-Nearest | Neighbors | Centroid | Machines | Predictor |
| id | label | Correct? | Correct? | Neighbor | Correct? | Correct? | Correct? | Correct? |

| | | | | | | | |
|---|---|---|---|---|---|---|---|
| Mean percent | 66 | 69 | 81 | 80 | 65 | 87 | 81 |
| of correct | | | | | | | |
| classification: | | | | | | | |

The following parameters can characterize performance of classifiers: Sensitivity, Specificity, Positive Predictive Value, Negative Predictive Value; Sensitivity is the probability for a class A sample to be correctly predicted as class A, Specificity is the probability for a non-class A sample to be correctly predicted as non-A, PPV is the probability that a sample predicted as class A actually belongs to class A, NPV is the probability that a sample predicted as non-class A actually does not belong to class A.

For each classification method and each class, these parameters are listed in the tables below

| | | | | |
|---|---|---|---|---|
| Class | Sensitivity | Specificity | PPV | NPV |

Performance of the 1-Nearest Neighbor Classifier:

| | | | | |
|---|---|---|---|---|
| Carc_HR_LR | 0.917 | 0.64 | 0.811 | 0.821 |
| Control | 0.64 | 0.917 | 0.821 | 0.811 |

Performance of the 3-Nearest Neighbors Classifier:

| | | | | |
|---|---|---|---|---|
| Carc_HR_LR | 0.964 | 0.52 | 0.771 | 0.897 |
| Control | 0.52 | 0.964 | 0.897 | 0.771 |

Performance of the Support Vector Machine Classifier:

| | | | | |
|---|---|---|---|---|
| Carc_HR_LR | 0.94 | 0.76 | 0.868 | 0.884 |
| Control | 0.76 | 0.94 | 0.884 | 0.868 |

### Example 7.2

### “Carcinoma & Polyps Vs Contr”—25 Greedy Pairs>1NN & SVM 78%

The following markers were identified according to this example:

List 4: ARHGAP21, ARHGEF1, ASB13, BCKDHA, BCS1L, C10orf76, C17orf28, TMEM98, C17orf90, AFP, AFP, CHD8, DEF8, EID1, RPA1, TACC2, ACTL6B, HMGN2, FASN, C10orf2, HAUS4, BTBD6, MRPL47, MTCH2, NARFL, NME2, NRBP2, NOA1, PDP1, PIK3R2, PSMA2, PSMC2, RAD1, RPL26, RPL37A, SBF2, SERPINA1, SLC4A3, MGAT4B, UBE2L3, UMPS, UROD, PAICS, VPS72 (includes EG:100001285), WDR13, ZNF668.

Alternatively to example 7.1 the “greedy pairs” strategy was used for class prediction, and it was possible to very efficiently build classifiers for distinguishing “Contr” versus “Carcinoma & polyps” (contrast 1). Using “25 greedy pairs” of features on arrays, the 1-Nearest Neighbor Predictor (1-NN) and the Support Vector Machines Predictor (SVM) enabled correct classification of 78% of samples.

Greedy pairs algorithm was used to select 25 pairs of genes. Repeated 1 times K-fold (K=20) cross-validation method was used to compute mis-classification rate.

Performance of classifiers during cross-validation.

| | | | | | Diagonal | | | | | Bayesian |
|---|---|---|---|---|---|---|---|---|---|---|
| | | | Mean | Compound | Linear | | | | Support | Compound |
| | | | Number | Covariate | Discriminant | | 3-Nearest | Nearest | Vector | Covariate |
| | Array | Class | of genes in | Predictor | Analysis | 1-Nearest | Neighbors | Centroid | Machines | Predictor |
| | id | label | classifier | Correct? | Correct? | Neighbor | Correct? | Correct? | Correct? | Correct? |
| Mean percent | | | | 70 | 71 | 78 | 75 | 69 | 78 | 77 |
| of correct | | | | | | | | | | |
| classification: | | | | | | | | | | |

| | | | | |
|---|---|---|---|---|
| Class | Sensitivity | Specificity | PPV | NPV |

Performance of the 1-Nearest Neighbor Classifier:

| | | | | |
|---|---|---|---|---|
| Carc_HR_LR | 0.929 | 0.52 | 0.765 | 0.812 |
| Control | 0.52 | 0.929 | 0.812 | 0.765 |

Performance of the 3-Nearest Neighbors Classifier:

| | | | | |
|---|---|---|---|---|
| Carc_HR_LR | 0.94 | 0.44 | 0.738 | 0.815 |
| Control | 0.44 | 0.94 | 0.815 | 0.738 |

### Example 7.3

### “Carc Vs. Contr”—25 Greedy Pairs>97% 1NN

The following markers were identified according to this example:

List 5: APBA3, C11orf80, CAMK1D, CCND1, CEP57, COL6A3, HSPA1A/HSPA1B, CSRNP1, CUL1, DDX19B, DENND5A, EHMT2, EIF3L, ERBB3 (includes EG:13867), TACC2, FBN1, HK1, IVNS1ABP, JMJD7-PLA2G4B, KIF3C, METTL2B, NAGLU, NCAN, NFKB2, NME2, PBX2, PDZD4, PLXNA1, PRKACA, PSMB6, RPL24, RPS7, RUVBL2, SGSH, SPRY1, TAGLN3, TNFAIP2, TSTA3, TTR, UMPS.

For contrast 2 the “greedy pairs” strategy was used for class prediction for the first 34 (17 carc; 17 contr) samples of runt, and it was possible to very efficiently build classifiers for distinguishing “Contr” versus “Carc”. Using “25 greedy pairs” of features on arrays, the 1-Nearest Neighbor Predictor (1-NN) enabled best correct classification of 97% of samples.

Greedy pairs algorithm was used to select 25 pairs of genes. Leave-one-out cross-validation method was used to compute mis-classification rate.

Performance of classifiers during cross-validation.

| | | | | | Diagonal | | | | | Bayesian |
|---|---|---|---|---|---|---|---|---|---|---|
| | | | Mean | Compound | Linear | | | | Support | Compound |
| | | | Number | Covariate | Discriminant | | 3-Nearest | Nearest | Vector | Covariate |
| | Array | Class | of genes in | Predictor | Analysis | 1-Nearest | Neighbors | Centroid | Machines | Predictor |
| | id | label | classifier | Correct? | Correct? | Neighbor | Correct? | Correct? | Correct? | Correct? |
| Mean percent | | | | 91 | 91 | 97 | 97 | 97 | 91 | 91 |
| of correct | | | | | | | | | | |
| classification: | | | | | | | | | | |

| | | | | |
|---|---|---|---|---|
| Class | Sensitivity | Specificity | PPV | NPV |

Performance of the 1-Nearest Neighbor Classifier:

| | | | | |
|---|---|---|---|---|
| Carcinoma | 0.941 | 1 | 1 | 0.944 |
| Control | 1 | 0.941 | 0.944 | 1 |

Performance of the 3-Nearest Neighbors Classifier:

| | | | | |
|---|---|---|---|---|
| Carcinoma | 0.941 | 1 | 1 | 0.944 |
| Control | 1 | 0.941 | 0.944 | 1 |

Performance of the Nearest Centroid Classifier:

| | | | | |
|---|---|---|---|---|
| Carcinoma | 0.941 | 1 | 1 | 0.944 |
| Control | 1 | 0.941 | 0.944 | 1 |

### Example 7.4

### “Carc Vs. Contr”—25 Greedy Pairs—80% 1NN

The following markers were identified according to this example:

List 6: A1BG, ACTR1B, ADCK3, AP3D1, ARAP1, CHST10, BCL6, TMEM98, C17orf90, AFP, AFP, C6orf136, COL8A2, CTNND2, DCAF15, GTF3A, ICAM3, IFNGR2, LMNA, LRRC4C, MAN2B1, MTA1, NPDC1, LOC440354, PMVK, POMGNT1, POTEE/POTEF, PPCDC, TSTA3, RBPJ, RPL7, SETD1B, SLMAP, SNTA1, STAB1, SUOX, TIAL1, TMCO7, TRIP12, TRPC4AP, ZCCHC14, ZNF589.

The “greedy pairs” strategy was used for class prediction of the first 34 (17 carc; 17 contr) samples processed in run2, and it was possible to very efficiently build classifiers for distinguishing “Contr” versus “Carc”. Using “25 greedy pairs” of features on arrays, the 1-Nearest Neighbor Predictor (1-NN) enabled best correct classification of 80% of samples.

Greedy pairs algorithm was used to select 25 pairs of genes. Leave-one-out cross-validation method was used to compute mis-classification rate.

Performance of classifiers during cross-validation.

| | | | | | Diagonal | | | | | Bayesian |
|---|---|---|---|---|---|---|---|---|---|---|
| | | | Mean | Compound | Linear | | | | Support | Compound |
| | | | Number | Covariate | Discriminant | | 3-Nearest | Nearest | Vector | Covariate |
| | Array | Class | of genes in | Predictor | Analysis | 1-Nearest | Neighbors | Centroid | Machines | Predictor |
| | Id | label | classifier | Correct? | Correct? | Neighbor | Correct? | Correct? | Correct? | Correct? |
| Mean percent | | | | 77 | 70 | 73 | 80 | 73 | 73 | 80 |
| of correct | | | | | | | | | | |
| classification: | | | | | | | | | | |

Performance of the 1-Nearest Neighbor Classifier:

| Class | Sensitivity | Specificity | PPV | NPV |
|---|---|---|---|---|
| Carcinoma | 0.867 | 0.6 | 0.684 | 0.818 |
| Control | 0.6 | 0.867 | 0.818 | 0.684 |

### Example 7.5

### Carc Vs Contr—25 Greedy Pairs—1NN 79%

The following markers were identified according to this example:

List 7: ACTL6B, ARHGEF10L, ATP5H, ATXN2, TMEM98, C17orf90, AFP, AFP, C18orf32, CTDP1, DEF8, DPYSL5, EIF4A2, TACC2, ACTL6B, PLEKHO1, EXOSC8, HAUS4, BTBD6, LRRC4C, MACF1, MAN2B1, MTCH2, MTSS1L, NARFL, NBEAL2, NHEJ1, OTUD5, PDP1, PKM2, PLAA, PLEKHA5, POLN, PRKCSH, RAD1, RPL37A, RSL1D1, RSL1D1, SAFB2, SERPINA1, SPRY1, TPX2, UROD, UROD, PAICS, VPS18.

The “greedy pairs” strategy was used for class prediction of all carc & contr samples, and it was possible to very efficiently build classifiers for distinguishing “Contr” versus “Carc”. Using “25 greedy pairs” of features on arrays, the 1-Nearest Neighbor Predictor (1-NN) enabled best correct classification of 79% of samples.

Greedy pairs algorithm was used to select 25 pairs of genes. Repeated 1 times K-fold (K=20) cross-validation method was used to compute mis-classification rate.

Performance of classifiers during cross-validation.

| | | | | | Diagonal | | | | | Bayesian |
|---|---|---|---|---|---|---|---|---|---|---|
| | | | Mean | Compound | Linear | | | | Support | Compound |
| | | | Number | Covariate | Discriminant | | 3-Nearest | Nearest | Vector | Covariate |
| | Array | Class | of genes in | Predictor | Analysis | 1-Nearest | Neighbors | Centroid | Machines | Predictor |
| | id | label | classifier | Correct? | Correct? | Neighbor | Correct? | Correct? | Correct? | Correct? |
| Mean percent | | | | 77 | 72 | 79 | 72 | 78 | 73 | 81 |
| of correct | | | | | | | | | | |
| classification: | | | | | | | | | | |

| | | | | |
|---|---|---|---|---|
| Class | Sensitivity | Specificity | PPV | NPV |

Performance of the 1-Nearest Neighbor Classifier:

| | | | | |
|---|---|---|---|---|
| Carcinoma | 0.898 | 0.68 | 0.733 | 0.872 |
| Control | 0.68 | 0.898 | 0.872 | 0.733 |

Performance of the 3-Nearest Neighbors Classifier:

| | | | | |
|---|---|---|---|---|
| Carcinoma | 0.898 | 0.54 | 0.657 | 0.844 |
| Control | 0.54 | 0.898 | 0.844 | 0.657 |

### Example 7.6

### “Carc Vs Contr”—Grid of Alpha; 1.25 Fold>SVM79%

The following markers were identified according to this example:

List 8: ABCA3, ACAA2, ACTL6B, ACTL6B, ADNP, AHCY, ALB, AMBRA1, APLP1, ARAP1, CHST10, ARAP2, ARHGAP21, ZNF259, ARHGAP44, ARHGEF1, ARHGEF10L, ARHGEF17, ASCL1, ATP5H, ATXN2, AXIN1, BCAS2, BCS1L, BGN, C10orf76, C17orf28, TMEM98, C17orf90, AFP, AFP, C18orf32, C20orf20, PECAM1, C5orf25, C6orf136, CCDC64, CCT8, CHD8, CLCN6, CLTA, COL3A1, CPNE6, CTDP1, CUL1, CYC1, DBI, DCTPP1, DDX41, DEF8, DOCK9, DOCK9, DPP3, DPYSL5, DYNC1H1, EEF1A1, EFTUD1, EHMT2, EHMT2, EID1, RPA1, EPC1, EPS8, TACC2, ETS1, ACTL6B, PLEKHO1, EXOSC8, FAM149A, FAM204A, FHOD1, FLII, FLNB, GNAO1, GNPTG, GP1BB, GPCPD1, GTDC1, C10orf2, HAUS4, HERC4, HK1, HMGA1, JMJD4, HNRNPK, BTBD6, HSPA9, HTRA1, IDS, IMP4, ITSN1, KAT8, KCNIP1, LCP1, LDOC1, LMF2, LMNA, HMGB2, LRRC4C, LSM12 (includes EG:124801), LTBP3, MACF1, MAPK10, MAPRE1, MAST2, MRPL47, MTCH1, MTCH2, MTSS1L, NARFL, NASP, NBEAL2, NHEJ1, NKTR, NME2, NRBP2, NOA1, NSMCE2, NUMA1, PAPLN, PATZ1, PDIA3, PDP1, PFKL, PI4KA, PIK3CD, PIK3R2, PKM2, PLAA, PKM2, PLAA, PLCG1, PLEKHA5, PLXNA1, PLXNB1, PLXND1, POMT1, PPP1R2, PRKCSH, PSMB1, PTPRO, QTRT1, RBM4, RBM4, RHOB, RNF40, RPL13, RPL26, RPL27, RPL37A, RPS10, MICAL1, RSL1D1, RSL1D1, KLHL23/PHOSPHO2-KLHL23, RTN4 (includes EG:57142), RUVBL2, SAFB2, SCARF2, SDCBP, SERPINA1, SERPINH1, SGSH, SLC4A3, DARS, SLC4A2, SLC4A3, SNRNP40, SPAG1, SPAG17, SPRY1, SPTLC1, STAU1, STK17A, MGAT4B, TIAM2, TMCC2, TMEM123 (includes EG:114908), TP53 (includes EG:22059), TP53 (includes EG:22059), TP53BP1, TPX2, TRIOBP, TSTA3, TTYH3, TUBGCP2, UBE2L3, UROD, UROD, PAICS, VPS18, WDR73, YARS, YARS, YPEL1, YY1, ZNF12, ZNF358, ZNF512B, ZNF668.

Using feature selection upon “optimization of the grid of alpha levels” and “1.25 fold change of median intensities between classes” 7 classifier lists using different numbers of markers by the specific classification model (at an optimized significance level) were identified.

Genes significantly different between the classes at the 0.01, 0.005, 0.001 and 0.0005 significance levels were used to build four predictors. The predictor with the lowest cross-validation mis-classification rate was selected. The best compound covariate classifier consisted of genes significantly different between the classes at the 0.001 significance level. The best diagonal linear discriminant analysis classifier consisted of genes significantly different between the classes at the 0.001 significance level. The best 1-nearest neighbor classifier consisted of genes significantly different between the classes at the 0.005 significance level. The best 3-nearest neighbors classifier consisted of genes significantly different between the classes at the 0.001 significance level. The best nearest centroid classifier consisted of genes significantly different between the classes at the 0.005 significance level. The best support vector machines classifier consisted of genes significantly different between the classes at the 0.005 significance level. The best Bayesian compound covariate classifier consisted of genes significantly different between the classes at the 5e-04 significance level.

Only genes with the fold-difference between the two classes exceeding 1.25 were used for class prediction. Repeated 1 times K-fold (K=20) cross-validation method was used to compute mis-classification rate.

Performance of classifiers during cross-validation.

| | | | | Diagonal | | | | | Bayesian |
|---|---|---|---|---|---|---|---|---|---|
| | | | Compound | Linear | | | | Support | Compound |
| | | | Covariate | Discriminant | | 3-Nearest | Nearest | Vector | Covariate |
| | Array | Class | Predictor | Analysis | 1-Nearest | Neighbors | Centroid | Machines | Predictor |
| | id | label | Correct? | Correct? | Neighbor | Correct? | Correct? | Correct? | Correct? |
| Mean percent | | | 75 | 74 | 74 | 75 | 73 | 79 | 77 |
| of correct | | | | | | | | | |
| classification: | | | | | | | | | |

| | | | | |
|---|---|---|---|---|
| Class | Sensitivity | Specificity | PPV | NPV |

Performance of the 1-Nearest Neighbor Classifier:

| | | | | |
|---|---|---|---|---|
| Carcinoma | 0.918 | 0.56 | 0.672 | 0.875 |
| Control | 0.56 | 0.918 | 0.875 | 0.672 |

Performance of the 3-Nearest Neighbors Classifier:

| | | | | |
|---|---|---|---|---|
| Carcinoma | 0.939 | 0.56 | 0.676 | 0.903 |
| Control | 0.56 | 0.939 | 0.903 | 0.676 |

### Example 7.7

### “Carc_HR vs Contr_LR” p<0.005>SVM 83%

The following markers were identified according to this example:

List 9: AAAS, ABCE1, ACAA2, ACAP1, ACIN1, ACTL6B, ADNP, ALB, APBB1IP, ARHGAP21, ARHGAP25, ARHGEF10L, ARHGEF17, ARID5A, ASAP1, ASB13, ATP5D, AXIN1, AZI2, BAG6, BCAS2, BCKDHA, BCLAF1, C10orf76, C17orf28, TMEM98, C17orf90, AFP, AFP, C18orf32, PECAM1, CDH10, CDK16, CLDN5, CLTA, CLTA, CLUAP1, CNPY3, CRABP1, CSF1R, CTDP1, CUL1, CYHR1, DCAF13, DDX27, DDX41, DEF8, DHX36, DLG5, DLX2, EDC3 (includes EG:315708), EEF1A1, EID1, EIF4A2, TACC2, EVI5L, ACTL6B, PLEKHO1, EXOSC4, EXOSC8, EXOSC8, FAM108A1, FAM149A, FAM192A, FAM209B, FAM59B, FBRS, FHOD1, GALE, GNAO1, GPCPD1, GPN1, GTF2B, HARS, HAUS4, HBP1, HERPUD1, HMG20B, HMGN2, JMJD4, HNRNPA0, HNRNPK, HNRNPR, HNRPLL, ELAC2, BTBD6, HTRA1, INA, INPP5K, KIAA0753, KPNB1, LAMA5, LAMB1, LARP1, LMTK2, LONP1, LRRC4C, MAGED2, MAST2, MCM6, MCM9, MGAT4B, MIIP, MYL6, MYO1D, MYO1D, MYO5A, NARFL, NCOR1, NDUFA13, NME2, NRBP2, NOA1, NUMA1, OTUD5, PAICS, PDIA3, LOC440354, PIK3CD, PJA1, PKM2, PLAA, PKP3, PLXNB1, PLXND1, POLE2, POLRMT, POMT1, PPP5C, PQBP1, PRDX1, PRKCSH, PSKH1, PSMA2, PSMB5, PSMD10, PTPRF, RAB3IL1, RBM4, RBM4, RBM4, RBM4, REC8 (includes EG:290227), RHBDD2, RHOB, RNF220, RNF40, RP9, RPL13, RPL17, RPL22, RPL37A, SAFB2, SERPINA1, SERPINH1, SETD2, SFTPB, DARS, SH3BP2, SLC25A20, SPOCK2, SPRY1, SPTAN1, SPTLC1, STMN4, STT3B, TBC1D9B, THOC3, TIAM2, TMCC2, TMEM57, TNFRSF6B, TP53BP1, TPM3, ZWINT, TTC5, TUBB, TUBB3, TUBGCP2, TWF1 (includes EG:19230), UBC, UBE2L3, UBE2N, ULK1, UROD, UROD, VARS, PAICS, VPS18, WASH1/WASH5P, WDR13, WDR73, WDR77, XAB2, YARS, YPEL1, YY1, ZFYVE1, ZNF133, ZNF410, ZNF668, ZNF695, ZXDC.

Using feature selection upon “Genes significantly different between the classes at 0.005 significance level” and “1.25 fold change of median intensities between classes” 7 classifier lists using different numbers of markers by the specific classification model were identified. The Support Vector Machines Predictor gave the best correct classification of 83% using 226 markers.

Genes significantly different between the classes at 0.005 significance level were used for class prediction. Repeated 1 times K-fold (K=20) cross-validation method was used to compute mis-classification rate.

Performance of classifiers during cross-validation.

| | | | | | Diagonal | | | | | Bayesian |
|---|---|---|---|---|---|---|---|---|---|---|
| | | | Mean | Compound | Linear | | | | Support | Compound |
| | | | Number | Covariate | Discriminant | | 3-Nearest | Nearest | Vector | Covariate |
| | Array | Class | of genes in | Predictor | Analysis | 1-Nearest | Neighbors | Centroid | Machines | Predictor |
| | id | label | classifier | Correct? | Correct? | Neighbor | Correct? | Correct? | Correct? | Correct? |
| Mean percent | | | | 69 | 67 | 73 | 69 | 69 | 83 | 78 |
| of correct | | | | | | | | | | |
| classification: | | | | | | | | | | |

Performance of the Support Vector Machine Classifier:

| Class | Sensitivity | Specificity | PPV | NPV |
|---|---|---|---|---|
| Carc_HR | 0.851 | 0.806 | 0.814 | 0.844 |
| Cont_LR | 0.806 | 0.851 | 0.844 | 0.814 |

### Example 7.8

### “Carc_HR Vs Contr_LR” 25 Greedy Pairs>SVM 80%

The following markers were identified according to this example:

List 10: ABCE1, ACAA2, APBB1IP, ARHGAP21, ARHGEF10L, BCKDHA, C10orf76, C18orf32, CDK16, COMMD9, DHX36, DLG5, EID1, ACTL6B, PLEKHO1, GTF2B, HAUS4, JMJD4, HNRNPR, INPP5K, LAMB1, LRRC4C, NARFL, PDIA3, PIK3CD, PJA1, PKM2, PLAA, PLXND1, PPP5C, PSKH1, RNF40, RPL22, RPL37A, SERPINA1, SPRY1, TBC1D9B, TIAM2, TMCC2, VARS, PAICS, VPS18, YPEL1, ZNF668.

The “greedy pairs” strategy was used for class prediction of all Carc_HR vs Contr_LR samples, and it was possible to very efficiently build classifiers for distinguishing classes. Using “25 greedy pairs” of features on arrays, the Support Vector Machines Predictor (SVM) enabled best correct classification of 80% of samples.

Greedy pairs algorithm was used to select 25 pairs of genes. Repeated 1 times K-fold (K=20) cross-validation method was used to compute mis-classification rate.

Performance of classifiers during cross-validation.

| | | | | | Diagonal | | | | | Bayesian |
|---|---|---|---|---|---|---|---|---|---|---|
| | | | Mean | Compound | Linear | | | | Support | Compound |
| | | | Number | Covariate | Discriminant | | 3-Nearest | Nearest | Vector | Covariate |
| | Array | Class | of genes in | Predictor | Analysis | 1-Nearest | Neighbors | Centroid | Machines | Predictor |
| | id | label | classifier | Correct? | Correct? | Neighbor | Correct? | Correct? | Correct? | Correct? |
| Mean percent | | | | 70 | 72 | 73 | 75 | 71 | 80 | 79 |
| of correct | | | | | | | | | | |
| classification: | | | | | | | | | | |

Performance of the Support Vector Machine Classifier:

| Class | Sensitivity | Specificity | PPV | NPV |
|---|---|---|---|---|
| Carc_HR | 0.821 | 0.776 | 0.786 | 0.812 |
| Cont_LR | 0.776 | 0.821 | 0.812 | 0.786 |

### Example 7.9

### “Carc_HR Vs Contr_LR” 40 Recursive Features>SVM 82%

The following markers were identified according to this example:

List 11: ACAA2, APBB1IP, ASAP1, PECAM1, IK, CDT1, CHCHD8, CPNE2, DTNBP1, TACC2, ACTL6B, GNAO1, GRK6, HDAC6, ELAC2, LRIG1, LRRC16B, LRRC4C, MYO1C, NME2, KPNA2, PDIA3, LOC440354, PRRT2, PSKH1, RPL24, SNTA1, STMN4, TIAL1, TMCC2, TRAPPC3, GPX4, UBC, YLPM1, ZCCHC11, ZNF232.

Alternatively to the “greedy pairs” strategy for class prediction of all Carc_HR vs Contr_LR samples, the “Recursive feature” extraction strategy was used and selection of 40 features enabled 82% correct classification using the SVM method.

Recursive Feature Elimination method was used to select 40 genes. Repeated 1 times K-fold (K=20) cross-validation method was used to compute mis-classification rate.

Performance of classifiers during cross-validation.

| | | | | | Diagonal | | | | | Bayesian |
|---|---|---|---|---|---|---|---|---|---|---|
| | | | Mean | Compound | Linear | | | | Support | Compound |
| | | | Number | Covariate | Discriminant | | 3-Nearest | Nearest | Vector | Covariate |
| | Array | Class | of genes in | Predictor | Analysis | 1-Nearest | Neighbors | Centroid | Machines | Predictor |
| | id | label | classifier | Correct? | Correct? | Neighbor | Correct? | Correct? | Correct? | Correct? |
| Mean percent | | | | 78 | 74 | 79 | 74 | 73 | 82 | 79 |
| of correct | | | | | | | | | | |
| classification: | | | | | | | | | | |

Performance of the Support Vector Machine Classifier:

| Class | Sensitivity | Specificity | PPV | NPV |
|---|---|---|---|---|
| Carc_HR | 0.806 | 0.836 | 0.831 | 0.812 |
| Cont_LR | 0.836 | 0.806 | 0.812 | 0.831 |

### Example 7.10

### “Carc, HR, LR, Contr”—SVM>85%

The following markers were identified according to this example:

List 12, part a): ABCE1, ACAA2, AFMID, AKR1C4, AKR1C4, AKT2, AP2A1, BTBD7, APRT, ARHGEF10L, BMS1P5, C17orf90, AFP, CHD3, CLU, COPS6, CTC1, DCAF11, DLG5, ELP2, EPC1, FIBP, FN3K, GBP2 (includes EG:14469), GMPPB, HIPK3, IL6ST, INTS1, ITGAL, LRIG1, MTA2, MVD, MYO5A, NKRF, NOMO1 (includes others), OTUB1, PDE4D, PER1, PHF3, PLXNA3, POR, PSKH1, RABEPK, RNF40, RPL22, SBF1, SEMA3F, SEPT1, SERPINB9, SGSH, SPRN, SPSB3, TAGLN3, TBC1D9B, TBCB, TP53 (includes EG:22059), TSC2, TUBB6, USP39, VARS, WDR63, ZNF277, ZNF672.
List 12, part b): ABCA2, ABCE1, ACAA2, ACAP1, ACD, ACSL3, ACTB, ADCY1, ADRBK1, AGPAT6, AHSG, AKR1B1, AKT1, ALB, ANAPC5, IGSF9, ANXA6, AP2A1, BTBD7, APBA3, APRT, ARF4, ARF5, ARHGAP25, ARHGEF2, ARMCX1, ASB13, ATP5D, ATP8B3, BCLAF1, BCS1L, BMS1P5, BSDC1, BTBD11, C18orf21, C18orf32, PECAM1, C8orf33, C8orf33, CALR, CAPN2, CAPN2, NDUFB7, CCDC64, CCDC88B, CCL28, CCT8, CDC37, CDK16, CDK5RAP3, CHID1, CNPY3, COMMD9, COX7A2L, CPNE1, CPNE5, CRABP1, CSF1R, CSNK2B, CTBP2, CTBP2, DCAF10, DCTN3, DCTPP1, DDIT4, DDX27, DDX41, DDX56, DEF8, DHX8, DHX8, DLG5, DLX2, DPP3, DRAP1, DSE, DVL1, EEF1A1, EEF1A1, EEF1A1, EIF2B5, RPA1, ELMO1, ELP3, EPN1, EPN1, TACC2, ERP44, ESYT1, ACTL6B, MEAF6, FAM209B, FAM59B, KIAA1109, FAM65A, FCHSD1, FLII, FNTA, FXYD6, G3BP2, GAL3ST4, GGA1 (includes EG:106039), GHITM, GIT1 (includes EG:216963), GOLGA4, GSK3A, GTF3C5, HLA-A, HMGA1, HMGCL, HMGN2, JMJD4, HNRNPH1, HSD17B4, HSP90B1, HSPA1A/HSPA1B, IFRD1, INPP5K, JAG1, KCTD13, KHSRP, KIAA1244, KIF19, LAMP1, LCP1, LMTK2, LOC285463, LPCAT1, LSM14A, LTBP3, MAF1 (includes EG:315093), MAP1A, TCF19, MAPK8IP1, MAPK8IP3, MAST2, MEAF6, MGAT4B, MGAT4B, MIB2, MIIP, MLL4, MPI, MRPS11, MTCH1, MTCH2, RPL17, MUC5AC/MUC5B, NARF, NARFL, NBEAL2, NCF1, NCS1, NDUFA13, NDUFB10, NDUFS2, NIPSNAP1, NME2, NME2, NOMO1 (includes others), NPDC1, NR4A1, NT5C, NTAN1, NUMA1, ODC1, OLFML2A, ALB, PAICS, PCDH9, PDIA3, PI4KA, PIK3CD, PJA1, PKM2, PLAA, PKM2, PLAA, PLCE1, PLXNA3, PLXND1, PPID, PPM1F, PPP1R15A, PPP1R18, PRDX1, PRDX1, PRKCSH, PRKCZ, PRR3, PRRT1, PSAT1, PSKH1, PSMA2, PSMA2, PSME1, PSME1, ARHGDIA, PTPN12, PTPN23, PTPRS, BAD, PTPRS, BAD, HAPLN3, RANBP1, RBM15, RBM26, RBM4, RBM4, RC3H2, RHBDD2, RNF220, RPL10, RPL13, RPL22, RPL22, RPL26, RPL28, RPL37A, RPL7, RRP9, RTKN, RUSC1, SBF2, SCAF8, SCO1, SCOC, SDCBP, GRK6, SERBP1, SERPINA1, SETD2, SEZ6L2, SLC4A3, DARS, SLA, VIM, SMCHD1, SNIP1, SRA1, SRSF1, SRSF4, ST6GALNAC1, STOM, STX6, SUV420H1, SYS1 (includes EG:336339), TADA2B, MGAT4B, TLE1, TLE3, PXN, TMEM57, TMSB10/TMSB4X, TNFRSF25, TOE1, TOPORS, TPM3, ZWINT, TPM3, ZWINT, TRIM27, TTC3, TUBA1B, TUBA4A, TUBB, TUBGCP2, TWF2, UBB, UBE2N, UBXN1, UROD, TUBA1B, USP33, PAICS, VPS13C, VPS26A, WDR13, WDR35, WDR6, XYLT1, YPEL1, YY1, ZEB1, ZNF133, ZNF174, ZNF238, ZNF300, ZNF423, ZNF605, ZNF668.
List 12, part c): C10orf118, G6PD, GMIP, SNAPIN, HSPBAP1, RSL1D1, TCEA2, TPM3, ZWINT, TSPAN7.

Exemplifying the separation of all 4 different sample-classes, a “Binary tree” prediction analysis was conducted. As illustrated in that example the 1st classification node separates “Low Risk” from all the remaining classes using the markers of list 12, part a) at a “Mis-classification rate” (MCR) of 15.7%, then “controls” are distinguished from “Carcinoma & High Risk” by the markers of list 12, part b) (MCR=13.5%) and in the 3rd node “Carcinoma” are distinguished from “High Risk” by the markers of list 12, part c) (MCR=14.7%).

The markers of List 12, part a) can be used independently from the markers of list 12 parts b) and c) to distinguish LR from the combined group of cancer, healthy controls and HR polyps. Markers of list 12 parts b) and c) can be used to distinguish the indicated groups of classes (medical indications) but work best if the groups have been preselected by using the distinguishing markers of list 12a) in a previous step to remove the LR polyps from the question to be solved.

Binary Tree Classification algorithm was used. Feature selection was based on the univariate significance level (alpha=0.001). The support vector machine classifier was used for class prediction. There were 3 nodes in the classification tree. 10-fold cross-validation was performed.

Cross-validation error rates for a fixed tree structure shown below.

| | | | Mis- |
|---|---|---|---|
| | Group 1 | Group 2 | classification |
| Node | Classes | Classes | rate (%) |
| 1 | Carcinoma, Control, | Low Risk | 15.7 |
| | High Risk | | |
| 2 | Carcinoma, High Risk | Control | 13.5 |
| 3 | Carcinoma | High Risk | 14.7 |

Then the following parameters can characterize performance of classifiers: Sensitivity, Specificity, Positive Predictive Value (PPV), Negative Predictive Value (NPV) These parameters are listed in the table below

| Class | Sensitivity | Specificity | PPV | NPV |
|---|---|---|---|---|
| Carcinoma | 0.706 | 0.849 | 0.6 | 0.9 |
| Control | 0.556 | 0.923 | 0.714 | 0.857 |
| High Risk | 0.647 | 0.868 | 0.611 | 0.885 |
| Low Risk | 0.5 | 0.846 | 0.529 | 0.83 |

### Example 7.11

### “Carc, HR, LR, Contr”—SVM++

The following markers were identified according to this example:

List 13, part a): ABCA3, ADNP, AHCY, ALB, AMBRA1, ARHGAP21, ASCL1, ATP5H, AXIN1, BCS1L, BGN, C10orf76, C17orf28, TMEM98, C17orf90, AFP, AFP, C18orf32, PECAM1, C6orf136, CCDC64, CHD8, CLTA, COL3A1, CPNE6, CYC1, DCTPP1, DDX41, DEF8, DOCK9, DOCK9, DPP3, EEF1A1, EHMT2, EID1, RPA1, EPC1, EPS8, TACC2, ETS1, ACTL6B, EXOSC8, FLII, GPCPD1, C10orf2, HERC4, HMGA1, JMJD4, BTBD6, IDS, IMP4, ITSN1, KCNIP1, LCP1, LDOC1, HMGB2, LRRC4C, LSM12 (includes EG:124801), LTBP3, MACF1, MAST2, MRPL47, MTCH2, MTSS1L, NARFL, NASP, NBEAL2, NKTR, NME2, NRBP2, NOA1, NSMCE2, NUMA1, PATZ1, PDIA3, PDP1, PI4KA, PIK3CD, PIK3R2, PKM2, PLAA, PKM2, PLAA, PLCG1, PLXNB1, PLXND1, POMT1, PPP1R2, PRKCSH, PSMB1, RBM4, RBM4, RPL13, RPL26, RPL37A, SAFB2, SCARF2, SDCBP, SERPINA1, SLC4A3, DARS, SPRY1, SPTLC1, MGAT4B, TRIOBP, TUBGCP2, UBE2L3, UROD, UROD, PAICS, YARS, YARS, YPEL1, YY1, ZNF12, ZNF668.
List 13, part b): ACAA2, ARHGEF10L, C18orf32, CLTA, DBI, DCTPP1, EXOSC8, FLII, GNAO1, C10orf2, HNRNPK, LMNA, LRRC4C, LTBP3, MAST2, NARFL, NKTR, NUMA1, PIK3CD, PKM2, PLAA, PLXND1, RNF40, RPL26, RPL37A, RUVBL2, SGSH, TIAM2, TMCC2, TP53 (includes EG:22059), WDR73, YPEL1, YY1, ZNF512B.
List 13, part c): EFTUD1, ETS1, LRRC4C, NME2, PI4KA, PLEKHA5, QTRT1, RSL1D1, RSL1D1, TMEM183A.

For removal of any bias which might have been introduced upon the experimental design, the “Binary tree” prediction approach was repeated only to the experimental runs when all the 4 classes of samples were represented in two experiments. As expected classification success did improve; following numbers were obtained: As illustrated in the example the 1st classification node separates “Controls” from all the remaining classes using the markers of list 13, part a) at a “Mis-classification rate” (MCR) of 7.1%, then “Low Risk polyps” are distinguished from “Carcinoma & High Risk” by the markers of list 13, part b) (MCR=17.3%) and in the 3rd node “Carcinoma” are distinguished from “High Risk” by the markers of list 13, part c) (MCR=8.8%).

The markers of List 13, part a) can be used independently from the markers of list 13 parts b) and c) to distinguish health controls from the combined group of cancer, LR polyps and HR polyps. Markers of list 13 parts b) and c) can be used to distinguish the indicated groups of classes (medical indications) but work best if the groups have been preselected by using the distinguishing markers of list 13a) in a previous step to remove the healthy controls from the question to be solved.

Binary Tree Classification algorithm was used. Feature selection was based on the univariate significance level (alpha=0.05) The support vector machine classifier was used for class prediction. There were 3 nodes in the classification tree. 10-fold cross-validation was performed.

Cross-validation error rates for a fixed tree structure are shown below

| | | | Mis- |
|---|---|---|---|
| | Group 1 | Group 2 | classification |
| Node | Classes | Classes | rate (%) |
| 1 | Carcinoma, High Risk, | Control | 7.1 |
| | Low Risk | | |
| 2 | Carcinoma, High Risk | Low Risk | 17.3 |
| 3 | Carcinoma | High Risk | 8.8 |

| Class | Sensitivity | Specificity | PPV | NPV |
|---|---|---|---|---|
| Carcinoma | 0.647 | 0.774 | 0.478 | 0.872 |
| Control | 0.611 | 0.981 | 0.917 | 0.879 |
| High Risk | 0.647 | 0.849 | 0.579 | 0.882 |
| Low Risk | 0.444 | 0.865 | 0.533 | 0.818 |

### Example 7.12

### “Contr Vs LR”—40 Recursive Feature Extr>75%

The following markers were identified according to this example:

List 14: AP1B1, ARPP21, ATP2A3, BMS1P5, CCL28, CDK16, DCTPP1, DDX19A, ELAVL1, TACC2, ITGAL, LRPAP1, MAGED1, MAST2, MYO5A, NAV2, NEFL, PDP1, PEX19, PFKL, PKM2, PLAA, PLEKHM2, MED4 (includes EG:29079), PNCK, PPP2R5C, RNF10, ACO2 (includes EG:11429), SEPT1, TBC1D9B, TRMT2A, WDR5, ZNF277, ZNF514.

For example the “recursive feature” strategy was used for class prediction of all Contr vs LR samples, and it was possible to very efficiently build a classifier for distinguishing classes. Using 40 recursive features on arrays, all the predictors enabled correct classification of 75% of samples.

Recursive Feature Elimination method was used to select 40 genes. Repeated 1 times K-fold (K=20) cross-validation method was used to compute mis-classification rate.

Performance of classifiers during cross-validation.

| | | | | | Diagonal | | | | | Bayesian |
|---|---|---|---|---|---|---|---|---|---|---|
| | | | Mean | Compound | Linear | | | | Support | Compound |
| | | | Number | Covariate | Discriminant | | 3-Nearest | Nearest | Vector | Covariate |
| | Array | Class | of genes in | Predictor | Analysis | 1-Nearest | Neighbors | Centroid | Machines | Predictor |
| | id | label | classifier | Correct? | Correct? | Neighbor | Correct? | Correct? | Correct? | Correct? |
| Mean percent | | | | 75 | 75 | 75 | 75 | 75 | 75 | 75 |
| of correct | | | | | | | | | | |
| classification: | | | | | | | | | | |

| Class | Sensitivity | Specificity | PPV | NPV |
|---|---|---|---|---|
| Control | 0.833 | 0.667 | 0.714 | 0.8 |
| Low Risk | 0.667 | 0.833 | 0.8 | 0.714 |

### Example 7.13

### LR Vs HR 25 Greedy Pairs>SVM=80%

The following markers were identified according to this example:

List 15: ABCE1, AKAP17A, AP2A1, BTBD7 BMS1P5, CELSR1, CENPT, CHD3, CYB5R3, DLG5, FCHSD1, GTF2B, HIST1H2AC, HNRNPM, HNRNPR, ITGA5, JAG1, KIF21A, MAP4, NBPF11 (includes others), NDRG1, NDUFA13, NDUFS5, PHF3, PREP, RALGDS, REEP2, RNF213, RNF40, RPL17, RPL22, RUFY1, SEPT1, SETD2, SMCHD1, SORD, NAP1L1, SYS1 (includes EG:336339), TPM3, ZWINT, UBXN7, WDR62, ZNF672. The “greed pairs” strategy was used for class prediction of all LR vs HR samples, and a classifier for distinguishing classes was defined. Using 50 features on arrays, the nearest neighbor (NN) and SVM predictors enabled correct classification of 80% of samples.

Greedy pairs algorithm was used to select 25 pairs of genes. Repeated 1 times K-fold (K=20) cross-validation method was used to compute mis-classification rate.

Performance of classifiers during cross-validation.

| | | | | | Diagonal | | | | | Bayesian |
|---|---|---|---|---|---|---|---|---|---|---|
| | | | Mean | Compound | Linear | | | | Support | Compound |
| | | | Number | Covariate | Discriminant | | 3-Nearest | Nearest | Vector | Covariate |
| | Array | Class | of genes in | Predictor | Analysis | 1-Nearest | Neighbors | Centroid | Machines | Predictor |
| | id | label | classifier | Correct? | Correct? | Neighbor | Correct? | Correct? | Correct? | Correct? |
| Mean percent | | | | 77 | 71 | 80 | 80 | 77 | 80 | 76 |
| of correct | | | | | | | | | | |
| classification: | | | | | | | | | | |

| | | | | |
|---|---|---|---|---|
| Class | Sensitivity | Specificity | PPV | NPV |

Performance of the 1-Nearest Neighbor Classifier:

| | | | | |
|---|---|---|---|---|
| High Risk | 0.824 | 0.778 | 0.778 | 0.824 |
| Low Risk | 0.778 | 0.824 | 0.824 | 0.778 |

Performance of the 3-Nearest Neighbors Classifier:

| | | | | |
|---|---|---|---|---|
| High Risk | 0.824 | 0.778 | 0.778 | 0.824 |
| Low Risk | 0.778 | 0.824 | 0.824 | 0.778 |

Performance of the Support Vector Machine Classifier:

| | | | | |
|---|---|---|---|---|
| High Risk | 0.882 | 0.722 | 0.75 | 0.867 |
| Low Risk | 0.722 | 0.882 | 0.867 | 0.75 |

### Example 7.14

### LR Vs HR 19 Greedy Pairs>CCP, 1NN=83%

The following markers were identified according to this example:

List 16: ABCE1, AP2A1, BTBD7, CELSR1, CENPT, CYB5R3, DLG5, FCHSD1, GTF2B, HIST1H2AC, HNRNPM, HNRNPR, KIF21A, MAP4, NBPF11 (includes others), NDRG1, PHF3, PREP, REEP2, RNF213, RNF40, RPL17, RPL22, RUFY1, SMCHD1, SORD, NAP1L1, SYS1 (includes EG:336339), TPM3, ZWINT, UBXN7, WDR62, ZNF672.

Then the “recursive feature” strategy for class prediction of all Carc vs HR samples was used, and it was possible to very efficiently build a classifier for distinguishing classes. Using 40 recursive features on arrays, the CCVP, SVM and BCCVP predictor enabled correct classification of 85% of samples.

Greedy pairs algorithm was used to select 19 pairs of genes. Repeated 1 times K-fold (K=20) cross-validation method was used to compute mis-classification rate.

Performance of classifiers during cross-validation.

| | | | | | Diagonal | | | | | Bayesian |
|---|---|---|---|---|---|---|---|---|---|---|
| | | | Mean | Compound | Linear | | | | Support | Compound |
| | | | Number | Covariate | Discriminant | | 3-Nearest | Nearest | Vector | Covariate |
| | Array | Class | of genes in | Predictor | Analysis | 1-Nearest | Neighbors | Centroid | Machines | Predictor |
| | id | label | classifier | Correct? | Correct? | Neighbor | Correct? | Correct? | Correct? | Correct? |
| Mean percent | | | | 74 | 71 | 83 | 80 | 77 | 77 | 76 |
| of correct | | | | | | | | | | |
| classification: | | | | | | | | | | |

Performance of the 1-Nearest Neighbor Classifier:

| Class | Sensitivity | Specificity | PPV | NPV |
|---|---|---|---|---|
| High Risk | 0.882 | 0.778 | 0.789 | 0.875 |
| Low Risk | 0.778 | 0.882 | 0.875 | 0.789 |

### Example 7.15

### “Carc Vs HR”—40 Recursive Feature Extr>SVM 85%

The following markers were identified according to this example:

List 17: ABCF1, ASNA1, SCHIP1, CCDC94, CCDC94, CELSR1, CLEC3B, COBRA1, ECHS1, EDARADD, ETS1, FAM114A2, KIAA1109, FAM65A, FLNA, GMIP, INTS1, IRF3, KDM3B, LARP1, METTL3, PBXIP1, PRKD2, PSMB5, QTRT1, RPL26, RSL1D1, RSL1D1, SCHIP1, SHF, SPINT1, SPTBN1, SPTBN1, UBE2Q1, WDR73.

Then the “recursive feature” strategy for class prediction of all Carc vs HR samples was used, and it was possible to very efficiently build a classifier for distinguishing classes. Using 40 recursive features on arrays, the CCVP, SVM and BCCVP predictor enabled correct classification of 85% of samples.

Recursive Feature Elimination method was used to select 40 genes. Repeated 1 times K-fold (K=20) cross-validation method was used to compute mis-classification rate.

Performance of classifiers during cross-validation.

| | | | | | Diagonal | | | | | Bayesian |
|---|---|---|---|---|---|---|---|---|---|---|
| | | | Mean | Compound | Linear | | | | Support | Compound |
| | | | Number | Covariate | Discriminant | | 3-Nearest | Nearest | Vector | Covariate |
| | Array | Class | of genes in | Predictor | Analysis | 1-Nearest | Neighbors | Centroid | Machines | Predictor |
| | id | label | classifier | Correct? | Correct? | Neighbor | Correct? | Correct? | Correct? | Correct? |
| Mean percent | | | | 85 | 82 | 74 | 79 | 79 | 85 | 85 |
| of correct | | | | | | | | | | |
| classification: | | | | | | | | | | |

| | | | | |
|---|---|---|---|---|
| Class | Sensitivity | Specificity | PPV | NPV |

Performance of the Compound Covariate Predictor Classifier:

| | | | | |
|---|---|---|---|---|
| Carcinoma | 0.941 | 0.765 | 0.8 | 0.929 |
| High Risk | 0.765 | 0.941 | 0.929 | 0.8 |

Performance of the Support Vector Machine Classifier:

| | | | | |
|---|---|---|---|---|
| Carcinoma | 0.882 | 0.824 | 0.833 | 0.875 |
| High Risk | 0.824 | 0.882 | 0.875 | 0.833 |

Performance of the Bayesian Compound Covariate Classifier:

| | | | | |
|---|---|---|---|---|
| Carcinoma | 0.941 | 0.706 | 0.762 | 0.923 |
| High Risk | 0.706 | 0.941 | 0.923 | 0.762 |

### Example 7.16

### “Carc Vs HR”/20 Greedy Pairs>SVM=85%

The following markers were identified according to this example:

List 18: ADRBK1, ATCAY, C10orf118, CELSR1, L1CAM, CYB5R3, G6PD, GMIP, GTF2I, HNRNPA1, SNAPIN, LRSAM1, MUM1, NASP, NFKBID, NR4A1, PGK1, PHF19, RBM4, RPL27, RSL1D1, RSL1D1, ACO2 (includes EG:11429), SF3B1, TALDO1, TCEA2, THBS1, TPM3, ZWINT, TSPAN7, ZMIZ.

Again using the “greed pairs” strategy for class prediction of Carc vs HR samples, 40 features on arrays, enabled correct classification of 85% of samples by the SVM classifier.

Greedy pairs algorithm was used to select 20 pairs of genes. Repeated 1 times K-fold (K=20) cross-validation method was used to compute mis-classification rate.

Performance of classifiers during cross-validation.

| | | | | | Diagonal | | | | | Bayesian |
|---|---|---|---|---|---|---|---|---|---|---|
| | | | Mean | Compound | Linear | | | | Support | Compound |
| | | | Number | Covariate | Discriminant | | 3-Nearest | Nearest | Vector | Covariate |
| | Array | Class | of genes in | Predictor | Analysis | 1-Nearest | Neighbors | Centroid | Machines | Predictor |
| | id | label | classifier | Correct? | Correct? | Neighbor | Correct? | Correct? | Correct? | Correct? |
| Mean percent | | | | 71 | 74 | 79 | 76 | 76 | 85 | 75 |
| of correct | | | | | | | | | | |
| classification: | | | | | | | | | | |

Performance of the Support Vector Machine Classifier:

| Class | Sensitivity | Specificity | PPV | NPV |
|---|---|---|---|---|
| Carcinoma | 0.882 | 0.824 | 0.833 | 0.875 |
| High Risk | 0.824 | 0.882 | 0.875 | 0.833 |

### DWD Transformed Data

In analogy to the “Class-prediction analyses” and examples depicted after Quantil-Normalization (QNORM) of protein-chip data, DWD-transformed data were similarly analyzed.

### Example 7.17

Healthy vs diseased samples (Carc-HR-LR) comprising carcinoma and patients with polyps could be distinguished by a 1-Nearest Neighbour classifier at 83% correct classification using 25 greedy pairs for feature selection.

The following markers were identified according to this example:

List 19: ADH5 (includes EG:11532), ALDOA, ARHGEF1, ATXN10, BIN3, C17orf28, TMEM98, C17orf90, AFP, AFP, CDK16, COL3A1, CYC1, DCTPP1, DEF8, EIF4A2, RPA1, TACC2, ACTL6B, FAM160A2, LAMB3, LMTK2, LRRC4C, LTBP3, MTCH2, PAPLN, PDIA3, PDP1, PI4KA, PKM2, PLAA, PLCG1, PPP1R2, RBM4, RPL37A, SLC4A3, SPRY1, MGAT4B, ST3GAL3, TSTA3, UROD, USP7, PAICS, VASP, VPS18, ZNF668.

### Example 7.18

Healthy vs diseased samples (Carc-HR-LR) comprising carcinoma and patients with polyps could be distinguished by a Nearest Centroid Classifier at 83% correct classification using 40 greedy pairs for feature selection.

The following markers were identified according to this example:

List 20: ACADVL, ADH5 (includes EG:11532), AGFG1, ALDOA, ARHGEF1, ASB13, ATXN10, BIN3, C17orf28, TMEM98, C17orf90, AFP, AFP, CCNI, CDK16, COL3A1, CPLX1, CPNE6, CTDP1, CYC1, DCTPP1, DEF8, EIF4A2, RPA1, EPRS, TACC2, ACTL6B, FAM160A2, JMJD4, BTBD6, IMP4, LAMB3, LCP1, LMO7, LMTK2, LRRC4C, LTBP3, MRPL47, MRPS11, MTCH2, MTSS1L, NARFL, NBEAL2, PAPLN, PDIA3, PDP1, PI4KA, PKM2, PLAA, PLCG1, PLXND1, PPP1R2, PPP4R1, RBM4, RBM4, RPL26, RPL37A, SERPINA1, SF3B4, SLC4A3, SLC25A29, SNRNP40, SPRY1, SSRP1, MGAT4B, TMUB2, ST3GAL3, TSTA3, UROD, USP7, PAICS, VASP, VPS18, YARS, ZNF410, ZNF668.

### Example 7.19

Controls vs Carcinomas of a subset of 34 samples conducted in the same analyses run could be distinguished by a Compound Covariate, Support Vector Machines, and Bayesian Compound Covariate Predictor classifier at 94% correct classification using 25 greedy pairs for feature selection.

The following markers were identified according to this example:

List 21: ACAA2, ASB13, ATXN2, CAMK1D, CCL28, CKM, COL3A1, CUL1, CUL9, DCTPP1, DDX54, DOCK10, EEF1A1, TACC2, GSTP1, HMG20B, HMGN2, HNRNPK, INTS7, ISG15, LIMD2, LMNA, LPCAT1, MACF1, NASP, KPNA2, PAPLN, PBRM1, PDZD4, PHACTR3, PLEKHA5, PLXND1, PRDM2, RNF220, SGSH, SPRY1, ST6GALNAC1, TSTA3, TTYH3, TUBGCP2, UBB, ZNF174, ZNF410.

### Example 7.20

Controls vs Carcinomas of the entire study samples could be distinguished by a Compound Covariate Predictor classifier at 87% correct classification using 25 greedy pairs for feature selection.

The following markers were identified according to this example:

List 22: ACTL6B, ARHGEF10L, ARHGEF17, ATXN2, TMEM98, C17orf90, AFP, AFP, COL3A1, CPLX1, CYC1, DCTPP1, DOCK9, EIF4A2, ERBB3 (includes EG:13867), TACC2, ACTL6B, PLEKHO1, FAM204A, GEMIN2, GPSM1, HAUS4, IMP4, ISG15, LIMD2, LRRC4C, MACF1, MTCH2, NBEAL2, KPNA2, PAPLN, PKM2, PLAA, PLCG1, PLXND1, POTEE/POTEF, PPP4R1, PSMB4, REV3L, RPL37A, RSL1D1, KLHL23/PHOSPHO2-KLHL23, RTN4 (includes EG:57142), SLC4A3, SPRY1, TMC8, TRIOBP, TSTA3, TUBB3, UROD, PAICS, VPS18, ZNF410.

### Example 7.21

Controls vs Carcinomas of the entire study samples could be distinguished by a Compound Covariate Predictor classifier at 89% correct classification using 15 greedy pairs for feature selection.

The following markers were identified according to this example:

List 23: ACTL6B, ATXN2, TMEM98, C17orf90, AFP, AFP, COL3A1, CPLX1, DCTPP1, ERBB3 (includes EG:13867), TACC2, ACTL6B, GEMIN2, GPSM1, ISG15, LRRC4C, MACF1, MTCH2, NBEAL2, KPNA2, PAPLN, PLCG1, REV3L, RPL37A, RSL1D1, TRIOBP, TSTA3, UROD, PAICS, VPS18, ZNF410.

### Example 7.22

Controls vs Carcinomas of the entire study samples could be distinguished by a Support Vector Machines classifier at 83% correct classification using RECURSIVE feature extraction (n=40) for feature selection.

The following markers were identified according to this example:

List 24: ACTL6B, AFTPH, AKAP9, ARHGEF1, ASNA1, CCL28, CDH2, CSK, DCTPP1, DDX3Y, DNAJC10, EML2, GGA1 (includes EG:106039), GP1BB, HADHA, HMGN2, ISG15, KIAA0368, LRSAM1, MCM3AP, MTCH2, KPNA2, PKD1L1, POGZ, PRMT1, PRPF4B, QTRT1, SAMSN1, GRK6, SERBP1, TRAPPC3, ST3GAL3, YLPM1, YWHAZ.

### Example 7.23

“Controls and low risk poly-patients” vs “Carcinomas and high risk polyps” of the entire study samples could be distinguished by a 1-Nearest neighbor classifier (143 features) at 76% correct classification using a single-gene p-value of p<0.005 as cut-off for feature selection.

The following markers were identified according to this example:

List 25: ABCE1, ACAA2, ACTL6B, ADH5 (includes EG:11532), AKT2, IGSF9, ANXA6, ARHGEF10L, ARHGEF25, ASB13, ASPSCR1, AXIN1, PECAM1, C6orf136, CCL28, CERCAM, CLDN5, CLU, CPE (includes EG:12876), DBI, DCAF11, DCTPP1, DDR1, DDX27, DENR, DHX58, DLG5, EPC1, ACTL6B, PLEKHO1, EXOSC4, FAM213A, FBLL1, FBRS, FKBP15, FLII, FN1, GOSR1, GTF2B, HAPLN3, HARS, HAUS4, HEBP2, HLA-C, HNRNPK, HNRNPR, HNRPLL, HSPA8, INPP5K, INPPL1, INTS1, INTS1, ISG15, KDM4A, LAMB1, LCP1, LOC100132116, LRIG1, LRRC4C, LTBP3, LYSMD2, MAF1 (includes EG:315093), MRPL38 (includes EG:303685), MTCH1, MTCH2, RPL17, MYO5A, NARF, NBEAL2, NBR1, NDUFB10, KPNA2, OTUD1, PABPC1, PAPLN, PI4KA, PIK3IP1, PKD1L1, PKP3, PLXNA3, PLXND1, POMT1, PPP1R13B, PPP4R1, PRDX5, PRRT1, PSKH1, PSMB4, REC8 (includes EG:290227), RGS19, RHBDD2, RPL4, RNF40, RPL13, RPL17, RPL22, RPN1, SAT2, SERPINH1, SGSH, SH3BP2, SLC25A20, SMCHD1, STMN4, TBC1D9B, TBCB, TIAM2, TMC8, TMCC2, TPM3, ZWINT, TRAPPC4, TSTA3, UBE2N, USP48, VARS, VPS18, ZFP14, ZNF133, ZNF410, ZNF668, ZNF672.

### Example 7.24

“Controls and low risk poly-patients” vs “Carcinomas and high risk polyps” of the entire study samples could be could be distinguished by a Diagonal Discriminant Analyses Predictor classifier at 81% correct classification using 25 greedy pairs for feature selection.

The following markers were identified according to this example:

List 26: ACAA2, ADH5 (includes EG:11532), ARHGEF10L, ASPSCR1, TMEM98, C17orf90, AFP, AFP, COX7A2L, DCTPP1, DENR, DLG5, EIF4A2, EPC1, ACTL6B, PLEKHO1, FLII, HLA-C, HNRNPA2B1, HNRNPR, INPP5K, ISG15, LRRC4C, LTBP3, MAF1 (includes EG:315093), MTCH2, KPNA2, PAPLN, LOC440354, PLXND1, RGS19, RNF40, RPL22, SPRY1, SRRM2, TBC1D9B, TMC8, TSTA3, TUBB3, PAICS, VPS18, ZNF410, ZNF668, ZNF672.

### Example 7.25

Exemplifying the separation of all 4 different sample-classes, a “Binary tree” prediction analysis was conducted.

The following markers were identified according to this example:

List 27, part a): AARS, ABCA2, ACADVL, ACSL3, ACTB, ADCY1, ADH5 (includes EG:11532), AFTPH, AGFG1, AGPAT6, AHSG, AKR1C4, AKR1C4, AKT1, ALDOA, ANAPC5, IGSF9, ANTXR2, ANXA6, AP2A1, BTBD7, APBA3, APP, ARF4, ARF5, GDAP1L1, ASB13, ASMTL, ATP5D, ATXN10, ATXN10, BCS1L, BRD3, BTBD11, C10orf76, C17orf90, AFP, C18orf21, PECAM1, CALR, CAPN2, CARM1, NDUFB7, CCDC109B, CCDC136, CCL28, CDC37, CDK16, CDR1, CHD1L, CHGA, COPS3, COX7A2L, CPNE1, CPNE6, CSNK2B, CTSD, DCTN3, DCTPP1, DDB1, DDR1, DDX23, DDX41, DDX56, DEF8, DENND3, ARHGEF40, DPP3, EEF1A1, EEF1A1, EEF1A1, EHMT2, EIF2B5, RPA1, EPHB6, EPN1, EPRS, TACC2, ESYT1, ARPC1A, FAM193A, ARPC1A, FAM193A, FAM32A, FLII, FLNA, GAL3ST4, GGA1 (includes EG:106039), GHITM, GOLGA4, GSK3A, GTPBP3, C10orf2, HES6, HLA-C, HMGA1, HMGCL, HMGN2, JMJD4, HSD17B4, HSP90AB1, IDH3B, IGHA1, KAT5, KAT7, KCTD13, KHSRP, KIF19, KIFAP3, LAMB3, LCMT1, LCP1, LDLR, LMTK2, LTBP3, MACF1, TCF19, MAP1S, MAPK3, MAPK7, MARS, MATR3, MEAF6, MGAT4B, MIIP, MLL4, MTCH1, RPL17, NARG2, NBEAL2, NDUFB10, NDUFS2, NES, NLRC5, NME2, NOL12, NOMO1 (includes others), NPDC1, NRBP2, NOA1, OGT, ALB, PAICS, PALM, PCDH9, PDIA3, PDP1, PHF8, PI4KA, PKM2, PLAA, PKM2, PLAA, PLCE1, PLCG1, PLXND1, PODXL2, POGZ, POMT1, PPM1F, PPP1R2, PRDX1, PRKCZ, PRRT1, PSAT1, PSMA2, PSMD6, PSMD8, PSME1, PTCHD2, ARHGDIA, PTOV1, PTPRS, BAD, HAPLN3, RAI1, RBM15, RBM4, RBM4, RBM4, RHBDD2, RNF216, RNF220, RPL26, RPL27, RPL37A, RPL4, RPS21, RPS27A, KLHL23/PHOSPHO2-KLHL23, RTN4 (includes EG:57142), SCAF8, SCOC, SFXN1, SLC4A3, DARS, SH3GL1, SLA, SLC9A3R1, SMUG1, SNIP1, SRA1, SRA1, SRSF1, SSBP2, SSBP2, ST6GALNAC1, STIM2, STK25, STX6, SUV420H1, TADA2B, TARS, TBC1D7, MGAT4B, TERF2IP, HSPA5, TLE1, TMC8, TMED8, PXN, TNFRSF25, DSN1 (includes EG:100002916), TOE1, ST3GAL3, TRIM27, TTYH1, GPX4, TUBA1B, TUBA1B, TUBA1B, TUBB, TUBB, TUBGCP2, TWF2, UBB, UBE2N, UBXN1, UROD, TUBA1B, USP7, PAICS, VPS26A, WDR35, WDR6, WSB2, YARS, ZNF133, ZNF174, ZNF300, ZNF408, ZNF423, ZNF605, ZNF668.
List 27, part b): ABCE1, ACAA2, ACAT2, AFMID, AKAP17A, AKT2, ALAD, ALB, AP2A1, BTBD7, AP2A1, BTBD7, APRT, APRT, ZNF259, ARHGAP44, ARHGEF10L, ARHGEF25, ARRB2, CBR1, CCDC88B, CCL28, CLU, CORO7, CRABP1, CSK, CUL9, DCAF11, DCTPP1, DDIT4, DDR1, DDX19B, DDX27, DENR, DLG5, DPP9, DUS1L, DVL1, EEF1A1, EEF1A1, ELP2, EPC1, FCGBP, FLII, GLUL, GMPPB, HIPK3, HNRNPA0, HNRNPH1, HSPA8, IL2RG, IL6ST, INPP5K, INTS1, ISG15, ITGAL, LMAN1, LONP1, LRIG1, MAF1 (includes EG:315093), MCM3AP, MED4 (includes EG:29079), MGAT4B, MRPL27 (includes EG:287635), MTA2, MYH9, MYO5A, NDRG1, NDUFA13, NDUFB10, NISCH, KPNA2, OTUB1, PDE4D, PECAM1, PER1, PJA1, PLEKHG2, PLXNA3, PLXND1, POR, PPP1R13B, PRPF6, PRR3, PSKH1, RABEPK, HAPLN3, RBM26, SULT1A3/SULT1A4, RNF40, RPL17, RPL22, RPS6KA1 (includes EG:20111), RTKN, SEMA3F, SEPT1, GRK6, SERBP1, SERPINB9, SETD2, SGSH, SH3BP2, SLC9A3R1, SMCHD1, SNF8, SPRN, SRA1, SYS1 (includes EG:336339), TAX1BP1, TBC1D9B, TBCB, TMC8, TRPS1, TTC28, TUBB6, VARS, WDR63, XYLT1, ZNF133, ZNF408, ZNF672, ZNF784.

As illustrated in that example the 1st classification node separates “Controls” from all the remaining classes using the markers of list 27, part a) as classifier at a “Mis-classification rate” (MCR) of 7.1%, then “Low Risk” polyp-patients are distinguished from “Carcinoma & High Risk” by the markers of list 27, part b) (MCR=15.4%).

The markers of List 27, part a) can be used independently from the markers of list 27 part b) to distinguish health controls from the combined group of cancer, LR polyps and HR polyps. Markers of list 27 part b) can be used to distinguish the indicated groups of classes (medical indications; in this case LR vs. cancer plus HR) but work best if the groups have been preselected by using the distinguishing markers of list 27) in a previous step to remove the healthy controls from the question to be solved.

Binary Tree Classification algorithm was used. Feature selection was based on the univariate significance level (alpha=0.001). The support vector machine classifier was used for class prediction. There were 2 nodes in the classification tree. 10-fold cross-validation was performed.

Cross-validation error rates for a fixed tree structure shown below.

| | | | Mis- |
|---|---|---|---|
| | Group 1 | Group 2 | classification |
| Node | Classes | Classes | rate (%) |
| 1 | Carcinoma, High Risk, | Control | 7.1 |
| | Low Risk | | |
| 2 | Carcinoma, High Risk | Low Risk | 15.4 |

### Example 7.26

“Controls” vs “low risk poly-patients” of the entire study samples could be could be distinguished by a Nearest Centroid Predictor classifier at 89% correct classification using 25 greedy pairs for feature selection.

The following markers were identified according to this example:

List 28: AHSG, AKR1B1, BTBD11, CANX, CPNE1, DHX8, EHD4, RPA1, FBXO21, C10orf2, HMGCL, IKBKAP, ITGAL, LMTK2, MAPKAP1, MARS, MATR3, MUC2, NDUFS2, PDP1, PSMD6, PTPRS, BAD, RPL17, RPL37A, SEPT1, SLC4A3, STK17A, SUV420H1, TBC1D7, TUBA1B, TUBB6, WDR1.

### Example 7.27

“Low risk vs high risk polyp-patients” of the entire study samples could be could be distinguished by a Support Vector Machines classifier at 86% correct classification using 25 greedy pairs for feature selection.

The following markers were identified according to this example:

List 29: ABCE1, AKAP17A, AP2A1, BTBD7, CELSR1, CYB5R3, DLG5, DOC2B, EXOSC4, FCGBP, FLII, FNBP4, GTF2B, HERPUD1, HIST1H2AC, HNRNPM, HNRNPR, HSPA8, IK, MED4 (includes EG:29079), NBPF11 (includes others), PECAM1, PLEKHG2, PRDX5, PREP, REEP2, RNF10, RPL17, RPL22, RTKN, SEPT1, GRK6, SERBP1, SERBP1, NAP1L1, SYS1 (includes EG:336339), TBC1D9B, TBCB, XYLT1, ZNF672.

### Example 7.28

“Carcinoma vs high risk polyp-patients” of the entire study samples could be could be distinguished by a 1-Nearest Neighbor classifier at 68% correct classification using 25 greedy pairs for feature selection.

The following markers were identified according to this example:

List 30: ARHGEF10L, ATXN2, BCAM, C10orf118, C1orf174, C20orf20, CDK16, CELSR1, CLTA, CPSF1, L1CAM, ELP3, HMGN2, FASN, FBN3, G6PD, GNB1, SNAPIN, IDO1, LRSAM1, MUM1, NASP, PDE2A, PDZD4, PIK3IP1, POLR2E, PTPRF, PXN, REEP2, RPL27, RSL1D1, SF3B1, SLC25A6, SMTN, SRA1, TESK1, THBS3, TIPARP-AS1, TNXB, TPM3, ZWINT, TSPAN7, UBE2Q1, ZMIZ2, ZNF768.

### Example 7.29

“Carcinoma vs high risk polyp-patients” of the entire study samples could be could be distinguished by a Support Vector Machines classifier at 68% correct classification using recursive feature extraction (n=40) for feature selection.

The following markers were identified according to this example:

List 31: ACSS1, ASNA1, SCHIP1, CCDC94, CDH2, CELSR1, CHN2, DUSP4, ECHS1, EDARADD, EID1, FAM114A2, GLRX3, INTS1, NAP1L4, PFKL, PKM2, PLAA, PLEC, PRKD2, PRPF31, PSMB5, QTRT1, RABGGTA, SCHIP1, ACO2 (includes EG:11429), SERPINB9, SF3B1, SHF, SLC4A2, SPTBN1, SPTBN1, TESK1, TUBGCP6, UBE2Q1, USP5, ZNF358.

### Example 8

### Random Selection of Subsets of Classifiers

To exemplify the diagnostic potential of subsets from classifiers elucidated here, 10 markers were randomly chosen: Set A: UROD, ERCC5, RBM4, TCEAL2, PLXND1, ALDOA, LCP1, TMUB2, CTDP1, RPL3 (UniGene ID: Hs.575313); Set B: EXOC1, SGTA, PPP1R2, SERPINA1, YARS, ZNF410, CTDP1, SLC25A29, COL3A1, DCTPP1; from e.g. the 80 classifiers markers depicted after DWD transformation for distinguishing patients with carcinomas and polyps (Carc & HR & LR; n=84) versus Controls (n=50) (contrast 1). Then class prediction analysis was conducted and elucidated with the random sets A) 82%, and B) 75% correct classification by the Nearest Centroid Classifier (NCentr); —originally the NCentr classifier derived from the 80 genes enabled 83% correct classification. This confirms that classifiers from the present gene-lists have high potential for diagnostics, and that subsets of the classifiers panels as well as single classifiers are of high potential value for diagnostics.

To reconfirm this findings for the QNORM set of classifiers (the 2nd applied data normalization strategy in addition to DWD), the same 2 subsets were retested, of 10 random-classifiers on the QNORM data set. Class prediction analysis for distinguishing patients with carcinomas and polyps (Carc_HR_LR; n=84) versus Controls (n=50) (contrast 1) was again conducted. Random set A) enabled 83% correct classification, and random set B) 75% correct classification by the Nearest Centroid Classifier (NCentr); originally these 2 sets of 10 markers were only presented by some markers in the QNROM derived classifier.

Therefore subsets of the classifiers panels as well as single classifiers would be useful for classification and diagnostics, independent from initial normalization strategies upon which classifiers were generated.

Again for exemplification of the value of subsets of classifier markers from the panels defined in that application, the 50 classifier genes for distinguishing Cancer vs Controls were selected and randomly chosen subsets of 2-49 markers (x-axis) were selected 1000-times. Then the classification success obtained by that subsets using the nearest centroid classifier algorithm was calculated and median % misclassification is depicted on the y-axis (FIG. 2).

The 80 classifier markers for distinguishing Disease (Cancer & polyps) vs Controls were selected and randomly chosen subsets of 2-79 markers (x-axis) were selected 1000-times. Then the classification success obtained by that subset using the nearest centroid classifier algorithm was calculated and median % mis-classification is depicted on the y-axis (FIG. 3). Thus even single classifier markers are capable of correct classification (80% correct for set A)—corresponds to a median classification-error of 20%—FIG. 3; and e.g. 70% correct for set B) corresponds to a median classification-error of 30%—FIG. 3).

1.-15. (canceled)

16. A method of diagnosing colon cancer or the risk of colon cancer in a patient by detecting a set of marker proteins in a patient comprising detecting antibodies binding the marker protein or detecting the marker proteins or an antigenic fragment thereof in a sample of the patient.

17. The method of claim 16, further defined as a comprising detecting at least 2 or least 20% of the marker proteins selected from ACAA2, ACTL6B, ARHGEF1, ARHGEF10L, ASB13, ATXN2, C10orf76, C17orf28, TMEM98, C17orf90, AFP, AFP, COL3A1, CUL1, DCTPP1, DEF8, EIF4A2, RPA1, TACC2, ACTL6B, PLEKHO1, HAUS4, BTBD6, ISG15, LRRC4C, LTBP3, MACF1, MTCH2, NARFL, NBEAL2, KPNA2, PAPLN, PDIA3, PDP1, PI4KA, PKM2, PLAA, PLCG1, PLXND1, RNF40, RPL37A, SERPINA1, SLC4A3, SPRY1, TMCC2, TSTA3, UROD, PAICS, VPS18, ZNF410, ZNF668 in a patient comprising the step of detecting antibodies binding said marker proteins, or detecting said marker proteins or antigenic fragments thereof in a sample of the patient.

18. The method of claim 16, further defined as a comprising detecting at least 2 or least 20% of the marker proteins selected from the markers of List 3, 4, 5, 6, 7, 8, 9, 10, 11, 12, 13, 14, 15, 16, 17, 18, 19, 20, 21, 22, 23, 24, 25, 26, 27, 28, 29, 30, 31, or any combination thereof in a patient comprising the step of detecting antibodies binding said marker proteins, or detecting said marker proteins or antigenic fragments thereof in a sample of the patient.

19. The method of claim 18, wherein the combination is from Lists 4 and 20, wherein the markers are selected from ACADVL, ADH5, AGFG1, ALDOA, ARHGAP21, ARHGEF1, ASB13, ATXN10, BCKDHA, BCS1L, BIN3, C10orf76, C17orf28, TMEM98, C17orf90, AFP, AFP, CCNI, CDK16, CHD8, COL3A1, CPLX1, CPNE6, CTDP1, CYC1, DCTPP1, DEF8, EID1, EIF4A2, RPA1, TACC2, ACTL6B, FAM160A2, FASN, C10orf2, HAUS4, JMJD4, BTBD6, IMP4, LAMB3, LCP1, LMO7, LMTK2, LRRC4C, LTBP3, MRPL47, MRPS11, MTCH2, MTSS1L, NARFL, NBEAL2, NME2, NOA1, PAPLN, PDIA3, PDP1, PI4KA, PIK3R2, PKM2, PLAA, PLCG1, PLXND1, PPP1R2, PPP4R1, PSMA2, PSMC2, RAD1, RBM4, RBM4, RPL26, RPL37A, SBF2, SERPINA1, SF3B4, SLC4A3, SLC25A29, SNRNP40, SPRY1, SSRP1, MGAT4B, TMUB2, ST3GAL3, TSTA3, UBE2L3, UMPS, UROD, USP7, PAICS, VASP, VPS18, VPS72, WDR13, YARS, ZNF410, ZNF668.

20. The method of claim 19, further defined as a method of detecting cancer and/or pre-cancerous polyps.

21. The method of claim 18, wherein the combination is from Lists 4 and 20, wherein the markers are selected from lists 5, 6, 7, 21, 22, 23 and 24 and wherein the markers are selected from A1BG, ACAA2, ACTL6B, ACTR1B, ADCK3, AFTPH, AKAP9, AP3D1, APBA3, ARAP1, CHST10, ARHGEF1, ARHGEF10L, ARHGEF17, ASB13, ASNA1, ATP5H, ATXN2, BCL6, C11orf80, TMEM98, C17orf90, AFP, AFP, C18orf32, C6orf136, CAMK1D, CCL28, CCND1, CDH2, CEP57, CKM, COL3A1, COL6A3, COL8A2, HSPA1A/HSPA1B, CPLX1, CSK, CSRNP1, CTDP1, CTNND2, CUL1, CUL9, CYC1, DCAF15, DCTPP1, DDX19B, DDX3Y, DDX54, DEF8, DENND5A, DNAJC10, DOCK10, DOCK9, DPYSL5, EEF1A1, EHMT2, EIF3L, EIF4A2, EML2, ERBB3, TACC2, ACTL6B, PLEKHO1, EXOSC8, FAM204A, FBN1, GEMIN2, GGA1, GP1BB, GPSM1, GSTP1, GTF3A, HADHA, HAUS4, HK1, HMG20B, HNRNPK, BTBD6, ICAM3, IFNGR2, IMP4, INTS7, ISG15, IVNS1ABP, JMJD7-PLA2G4B, KIAA0368, KIF3C, LIMD2, LPCAT1, LRRC4C, LRSAM1, MACF1, MAN2B1, MCM3AP, METTL2B, MTA1, MTCH2, MTSS1L, NAGLU, NARFL, NASP, NBEAL2, NCAN, NFKB2, NHEJ1, NME2, KPNA2, NPDC1, OTUD5, PAPLN, PBRM1, PBX2, PDP1, PDZD4, PHACTR3, LOC440354, PKD1L1, PKM2, PLAA, PLCG1, PLEKHA5, PLXNA1, PLXND1, PMVK, POGZ, POLN, POMGNT1, POTEE/POTEF, PPCDC, PPP4R1, PRDM2, PRKACA, PRKCSH, PRMT1, TSTA3, PRPF4B, PSMB4, PSMB6, QTRT1, RAD1, RBPJ, REV3L, RNF220, RPL24, RPL37A, RPL7, RPS7, RSL1D1, RSL1D1, RTN4, RUVBL2, SAFB2, SAMSN1, GRK6, SERBP1, SERPINA1, SETD1B, SGSH, SLC4A3, SLMAP, SNTA1, SPRY1, ST6GALNAC1, STAB1, SUOX, TAGLN3, TIAL1, TMC8, TMCO7, TNFAIP2, TPX2, TRAPPC3, ST3GAL3, TRIOBP, TRIP12, TRPC4AP, TSTA3, TTR, TTYH3, TUBB3, TUBGCP2, UBB, UMPS, UROD, UROD, PAICS, VPS18, YLPM1, YWHAZ, ZCCHC14, ZNF174, ZNF410, ZNF589.

22. The method of claim 21, further defined as a method of detecting cancer.

23. The method of claim 18, wherein the combination is from Lists 9, 10, 11, 25, 26 and wherein the markers are selected from ABCE1, ACAA2, ACTL6B, ADH5, ANKRD36B, ANXA6, APBB1IP, ARHGAP21, ARHGEF10L, ARHGEF25, ASAP1, ASB13, ASPSCR1, AXIN1, BCKDHA, C10orf76, TMEM98, C17orf90, AFP, AFP, C18orf32, PECAM1, C6orf136, CCL28, IK, CDK16, CDT1, CERCAM, CHCHD8, CLDN5, CLU, COMMD9, COX7A2L, CPE, CPNE2, DBI, DCAF11, DCTPP1, DDR1, DDX27, DENR, DHX36, DHX58, DLG5, DTNBP1, EID1, EIF4A2, EPC1, TACC2, ACTL6B, PLEKHO1, EXOSC4, FAM213A, FBLL1, FBRS, FKBP15, FLII, FN1, GNAO1, GOSR1, GRK6, GTF2B, HAPLN3, HARS, HAUS4, HDAC6, HEBP2, HLA-C, JMJD4, HNRNPA2B1, HNRNPK, HNRNPR, HNRPLL, ELAC2, HSPA8, INPP5K, INPPL1, INTS1, INTS1, ISG15, KDM4A, LAMB1, LCP1, LOC100132116, LRIG1, LRIG1, LRRC16B, LRRC4C, LTBP3, LYSMD2, MAF1, MRPL38, MTCH1, MTCH2, RPL17, MYO1C, MYO5A, NARF, NARFL, NBEAL2, NBR1, NDUFB10, NME2, KPNA2, OTUD1, PABPC1, PAPLN, PDIA3, LOC440354, PI4KA, PIK3CD, PIK3IP1, PJA1, PKD1L1, PKM2, PLAA, PKP3, PLXNA3, PLXND1, POMT1, PPP1R13B, PPP4R1, PPP5C, PRDX5, PRRT1, PRRT2, PSKH1, PSMB4, REC8, RGS19, RHBDD2, RPL4, RNF40, RPL13, RPL17, RPL22, RPL24, RPL37A, RPN1, SAT2, SERPINA1, SERPINH1, SGSH, SH3BP2, SLC25A20, SMCHD1, SNTA1, SPRY1, SRRM2, STMN4, TBC1D9B, TBCB, TIAL1, TIAM2, TMC8, TMCC2, TPM3, ZWINT, TRAPPC3, TRAPPC4, TSTA3, TTYH1, GPX4, TUBB3, UBC, UBE2N, USP48, VARS, PAICS, VPS18, YLPM1, YPEL1, ZCCHC11, ZFP14, ZNF133, ZNF232, ZNF410, ZNF668, ZNF672.

24. The method of claim 23, further defined as a method of distinguishing a healthy condition involving low-risk polyps from a condition involving high-risk polyps and/or cancer.

25. The method of claim 18, wherein the combination is from Lists 4 and 20 and wherein the markers are selected from lists 14 and 28 and wherein the markers are selected from AHSG, AKR1B1, AP1B1, ARPP21, ATP2A3, BMS1P5, BTBD11, CANX, CCL28, CDK16, CPNE1, DCTPP1, DDX19A, DHX8, EHD4, RPA1, ELAVL1, TACC2, FBXO21, C10orf2, HMGCL, IKBKAP, ITGAL, LMTK2, LRPAP1, MAGED1, MAPKAP1, MARS, MAST2, MATR3, MUC2, MYO5A, NAV2, NDUFS2, NEFL, PDP1, PEX19, PFKL, PKM2, PLAA, PLEKHM2, MED4 (includes EG:29079), PNCK, PPP2R5C, PSMD6, PTPRS, BAD, RNF10, RPL17, RPL37A, ACO2 (includes EG:11429), SLC4A3, STK17A, SUV420H1, TBC1D7, TBC1D9B, TRMT2A, TUBA1B, TUBB6, WDR1, WDR5, ZNF277, ZNF514.

26. The method of claim 25, further defined as a method of diagnosing low-risk polyps.

27. The method of claim 16, further defined as a method of diagnosing cancer or pre-cancerous polyps.

28. The method of claim 27, further defined as a method of distinguishing between high-risk polyps and low-risk polyps, wherein said high-risk polyps comprise adenomatous villous, adenomatous tubulovillous, or co-occurrence of adenomatous tubular with tubulovillous polyps.

29. The method of claim 16, further defined as a method of distinguishing between combined groups of cancer and pre-cancerous states, including distinguishing healthy conditions from cancer plus pre-cancerous polyps (both high-risk and low-risk), healthy conditions from cancer, healthy conditions plus low-risk polyps from high-risk polyps, healthy conditions plus low-risk polyps from high-risk polyps plus cancer, healthy conditions from low-risk polyps, low-risk polyps from high-risk polyps, and/or high-risk polyps from cancer.

30. The method of claim 29, further comprising obtaining a positive result in distinguishing a healthy condition plus low-risk polyps from high-risk polyps plus cancer and conducting a further cancer test.

31. The method of claim 30, wherein the further cancer test comprises an endoscopy or a biopsy.

32. The method of claim 16, wherein detecting antibodies binding the marker protein or detecting the marker proteins or an antigenic fragment thereof comprises comparing a detection signal with detection signals of a healthy control, wherein an increase in the detection signal indicates colon cancer or said risk of colon cancer.

33. The method of claim 16, wherein detecting antibodies binding the marker protein or detecting the marker proteins or an antigenic fragment thereof comprises comparing a detection signal with detection signals of one or more known control samples of conditions selected from cancer, pre-cancerous polyps, and/or a healthy control, wherein the control samples are used to obtain a marker dependent signal pattern as indication classifier and the marker dependent signals of the patient is compared with and/or fitted onto said pattern thereby obtaining information of the diagnosed condition.

34. The method of claim 16, wherein detecting antibodies binding the marker protein or detecting the marker proteins or an antigenic fragment thereof comprises comparing a detection signal with detection signals of a cancerous or pre-cancerous polyp control and comparing said detection signals, wherein a detection signal from the sample of a patient in amplitude of at least 60% of the cancerous control indicates colon cancer or a risk of colon cancer, or detection signal in at least 60% of the used markers indicates colon cancer or a risk of colon cancer.

35. The method of claim 34, wherein a detection signal from the sample of a patient in amplitude of at least 80% of the cancerous control indicates colon cancer or a risk of colon cancer, or detection signal in at least 75% of the used markers indicates colon cancer or a risk of colon cancer.

36. A method of treating a patient having colon cancer or having a polyp with a high risk of developing colon cancer comprising detecting cancer or a polyp with a high risk of developing colon cancer with the method of claim 16 and removing said colon cancer or polyp.

37. A kit of diagnostic agents suitable to detect any marker or marker combination as defined in claim 16 wherein said diagnostic agents comprise marker proteins or antigenic fragments thereof suitable to bind antibodies in a sample.

38. The kit of claim 37, wherein the diagnostic agents are immobilized on a solid support.

39. The kit of claim 37, further comprising a computer-readable medium or a computer program product, comprising signal data for control samples with known conditions selected from cancer or a pre-cancerous polyp, and/or of healthy controls, and/or calibration or training data for analyzing said markers provided in the kit for diagnosing colon cancer or distinguishing conditions selected from healthy conditions, cancer, or the pre-cancerous polyps.

40. The kit of claim 37, comprising at most 3000 diagnostic agents, at most 2500 diagnostic agents, at most 2000 diagnostic agents, at most 1500 diagnostic agents, at most 1200 diagnostic agents, at most 1000 diagnostic agents, at most 800 diagnostic agents, at most 500 diagnostic agents, at most 300 diagnostic agents, at most 200 diagnostic agents, or at most 100 diagnostic agents.
